SEQUENCE LISTING (19 sequences)

SEQ ID NO: 1 - protein, 453 aa, Artificial Sequence
  Anti-CD20 REGN1979 Heavy Chain
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - protein, 449 aa, Artificial Sequence
  Anti-CD3 REGN1979 Heavy Chain
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 3 - protein, 214 aa, Artificial Sequence
  Common REGN1979 Light Chain
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 127 aa, Artificial Sequence
  Anti-CD20 REGN1979 HCVR
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSS

SEQ ID NO: 5 - protein, 123 aa, Artificial Sequence
  Anti-CD3 REGN1979 HCVR
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVAS

SEQ ID NO: 6 - protein, 108 aa, Artificial Sequence
  Common REGN1979 LCVR
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKR

SEQ ID NO: 7 - protein, 8 aa, Artificial Sequence
  Anti-CD20 REGN1979 HCDR1
GFTFNDYA

SEQ ID NO: 8 - protein, 8 aa, Artificial Sequence
  Anti-CD20 REGN1979 HCDR2
ISWNSDSI

SEQ ID NO: 9 - protein, 20 aa, Artificial Sequence
  Anti-CD20 REGN1979 HCDR3
AKDNHYGSGSYYYYQYGMDV

SEQ ID NO: 10 - protein, 8 aa, Artificial Sequence
  Anti-CD3 REGN1979 HCDR1
GFTFDDYT

SEQ ID NO: 11 - protein, 8 aa, Artificial Sequence
  Anti-CD3 REGN1979 HCDR2
ISWNSGSI

SEQ ID NO: 12 - protein, 16 aa, Artificial Sequence
  Anti-CD3 REGN1979 HCDR3
AKDNSGYGHYYYGMDV

SEQ ID NO: 13 - protein, 6 aa, Artificial Sequence
  Common REGN1979 LCDR1
QSVSSN

SEQ ID NO: 14 - protein, 3 aa, Artificial Sequence
  Common REGN1979 LCDR2
GAS

SEQ ID NO: 15 - protein, 9 aa, Artificial Sequence
  Common REGN1979 LCDR3
QHYINWPLT

SEQ ID NO: 16 - protein, 326 aa, Artificial Sequence
  IgG4 Heavy Chain Constant Region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 326 aa, Artificial Sequence
  IgG4 Heavy Chain Constant Region with H435R/Y436F Modifications
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNRFTQKSLSLSLGK

SEQ ID NO: 18 - protein, 329 aa, Artificial Sequence
  IgG1 Heavy Chain Constant Region
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - protein, 329 aa, Artificial Sequence
  IgG1 Heavy Chain Constant Region with H435R/Y436F Modifications
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNRFTQKSLSLSPGK

(19) United States
(12) Patent Application Publication
(10) Pub. No.: US 2021/0206870 A1
(43) Pub. Date: Jul. 8, 2021

(54) Stabilized Formulations Containing Anti-CD20 x Anti-CD3 Bispecific Antibodies

(71) Applicant: Regeneron Pharmaceuticals, Inc., Tarrytown, NY (US)

(72) Inventors: Yuan Cheng, Livingston, NJ (US); Xiaolin Tang, Old Tappan, NJ (US)

(21) Appl. No.: 17/116,699

(22) Filed: Dec. 9, 2020

Related U.S. Application Data
(60) Provisional application No. 62/946,207, filed on Dec. 10, 2019.

(51) Int. Cl. C07K 16/28; C07K 16/46

(52) U.S. Cl. CPC C07K 16/2887; A61K 2039/505; C07K 16/468; C07K 16/2809

(57) ABSTRACT

The present invention provides stable liquid pharmaceutical formulations comprising a human bispecific antibody that specifically binds to human CD20 and human CD3. In certain embodiments, the formulations contain, in addition to the bispecific antibody, a buffer, a surfactant, and a sugar. The pharmaceutical formulations of the present invention exhibit a substantial degree of antibody stability upon stress and storage.

### CROSS-REFERENCE TO RELATED APPLICATIONS

This application claims the benefit under 35 USC § 119(e) of U.S. Provisional Application No. 62/946,207, filed Dec. 10, 2019, which is incorporated herein by reference in its entirety for all purposes.

### REFERENCE TO A SEQUENCE LISTING

This application incorporates by reference the Sequence Listing submitted in Computer Readable Form as file 10664U501-Sequence.txt, created on Nov. 20, 2020 and containing 25,368 bytes.

### FIELD OF THE INVENTION

The present invention relates to the field of therapeutic antibody formulations. More specifically, the present invention relates to the field of pharmaceutical formulations comprising a human bispecific antibody that specifically binds to human CD20 and human CD3.

### BACKGROUND

Therapeutic macromolecules (e.g., antibodies) must be formulated in a manner that not only makes the molecules suitable for administration to patients, but also maintains their stability during storage and subsequent use. For example, therapeutic antibodies in liquid solution are prone to degradation, aggregation and/or undesired chemical modifications unless the solution is formulated properly. The stability of an antibody in liquid formulation depends not only on the kinds of excipients used in the formulation, but also on the amounts and proportions of the excipients relative to one another. Furthermore, other considerations aside from stability must be taken into account when preparing a liquid antibody formulation. Examples of such additional considerations include the concentration of antibody that can be accommodated by a given formulation, and the visual quality or appeal of the formulation. Thus, when formulating a therapeutic antibody, great care must be taken to arrive at a formulation that remains stable, contains an adequate concentration of antibody, and possesses other properties which enable the formulation to be conveniently administered to patients.

CD20 is a non-glycosylated phosphoprotein expressed on the cell membranes of mature B cells. CD3 is a homodimeric or heterodimeric antigen expressed on T cells in association with the T cell receptor complex (TCR) and is required for T cell activation.

Bispecific antibodies to human CD20 and human CD3 are one example of therapeutically relevant macromolecules that require proper formulation. Such antibodies are clinically useful for, e.g., the treatment of cancer (e.g., B cell cancers such as follicular lymphoma, diffuse large B-cell lymphoma, mantle cell lymphoma, marginal zone lymphoma, or other non-Hodgkin's lymphomas).

Although anti-CD20×anti-CD3 bispecific antibodies are known in the art (see, e.g., WO 2014/047231), there remains a need for pharmaceutical formulations comprising anti-CD20×anti-CD3 bispecific antibodies that are sufficiently stable and suitable for administration to patients.

### BRIEF SUMMARY OF THE INVENTION

Stable liquid pharmaceutical formulations comprising a bispecific anti-CD20×anti-CD3 antibody and one or more excipients, as well as kits and unit dosage forms comprising such formulations and uses thereof, are provided.

In one aspect, the present invention provides a stable liquid pharmaceutical formulation comprising: (a) a bispecific antibody comprising a first antigen-binding domain that binds specifically to human CD20 and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) comprising the amino acid sequence of SEQ ID NO: 4 and three light chain CDRs (LCDR1, LCDR2 and LCDR3) contained in a light chain variable region (LCVR) comprising the amino acid sequence of SEQ ID NO: 6, and the second antigen-binding domain comprises three heavy chain CDRs (A2-HCDR1, A2-HCDR2 and A2-HCDR3) contained in a heavy chain variable region (HCVR) comprising the amino acid sequence of SEQ ID NO: 5 and three light chain CDRs (LCDR1, LCDR2 and LCDR3) contained in a light chain variable region (LCVR) comprising the amino acid sequence of SEQ ID NO: 6; (b) a buffer comprising histidine; (c) an organic co-solvent comprising polysorbate; and (d) a stabilizer comprising a sugar; wherein the formulation has a pH of 5.8±0.3.

In one aspect, the present invention provides a stable liquid pharmaceutical formulation comprising: (a) a bispecific antibody comprising a first antigen-binding domain that binds specifically to human CD20 and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 7, 8 and 9, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 13, 14 and 15; (b) a buffer comprising histidine; (c) an organic co-solvent comprising polysorbate; and (d) a stabilizer comprising a sugar; wherein the formulation has a pH of 5.8±0.3.

In some embodiments, the antibody concentration is from 1 mg/ml±0.1 mg/ml to 200 mg/ml±20 mg/ml. In some embodiments, the antibody concentration is 2 mg/ml±0.2 mg/ml. In some embodiments, the antibody concentration is 20 mg/ml±2 mg/ml. In some embodiments, the antibody concentration is 80 mg/ml±8 mg/ml. In some embodiments, the antibody concentration is 100 mg/ml±10 mg/ml. In some embodiments, the antibody concentration is 160 mg/ml±16 mg/ml. In some embodiments, the antibody concentration is from about 2 mg/ml to about 100 mg/ml.

In some embodiments, the histidine buffer concentration is from 5 mM±1 mM to 15 mM±3 mM. In some cases, the histidine buffer concentration is 10 mM±1 mM. In some embodiments, the histidine buffer comprises L-histidine and L-histidine monohydrochloride monohydrate. In some cases, the L-histidine concentration is 3.65 mM±0.5 mM and the L-histidine monohydrochloride monohydrate concentration is 6.35 mM±0.5 mM. In some cases, the histidine buffer comprises 0.57±0.05 mg/ml L-histidine and 1.33±0.13 mg/ml L-histidine monohydrochloride monohydrate.

In some embodiments, the polysorbate concentration is from 0.01%±0.005% to 0.5%±0.25% w/v. In some cases, the polysorbate concentration is 0.1%±0.05% w/v. In some cases, the polysorbate concentration is 0.1%±0.01% w/v. In some embodiments, the polysorbate is polysorbate 80.

In some embodiments, the sugar is sucrose. In some cases, the sucrose concentration is from 5%±1% to 20%±4% w/v. In some cases, the sucrose concentration is from 8%±0.5% to 12%±0.5% w/v. In some embodiments, the sucrose concentration is 10%±1% w/v.

In some embodiments, the pharmaceutical composition comprises: (a) 2 mg/ml±0.2 mg/ml antibody, (b) from 5 mM±1 mM to 15 mM±3 mM histidine buffer, (c) from 0.01%±0.005% to 0.5%±0.25% w/v polysorbate, and (d) from 5%±1% to 20%±4% w/v sucrose, at pH 5.8±0.3.

In some embodiments, the pharmaceutical composition comprises: (a) 2 mg/ml±0.2 mg/ml antibody, (b) 10 mM±1 mM histidine buffer, (c) 0.1%±0.01% w/v polysorbate, and (d) 10%±1% w/v sucrose, at pH 5.8±0.3.

In some embodiments, the pharmaceutical composition comprises: (a) 2 mg/ml±0.2 mg/ml antibody, (b) 3.65 mM±0.2 mM L-histidine, (c) 6.35 mM±0.2 mM L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 2 mg/ml±0.2 mg/ml antibody, (b) 0.57 mg/ml±0.05 mg/ml L-histidine, (c) 1.33 mg/ml±0.13 mg/ml L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 20 mg/ml±2 mg/ml antibody, (b) from 5 mM±1 mM to 15 mM±3 mM histidine buffer, (c) from 0.01%±0.005% to 0.5%±0.25% w/v polysorbate, and (d) from 5%±1% to 20%±4% w/v sucrose, at pH 5.8±0.3.

In some embodiments, the pharmaceutical composition comprises: (a) 20 mg/ml±2 mg/ml antibody, (b) 10 mM±1 mM histidine buffer, (c) 0.1%±0.01% w/v polysorbate, and (d) 10%±1% w/v sucrose, at pH 5.8±0.3.

In some embodiments, the pharmaceutical composition comprises: (a) 20 mg/ml±2 mg/ml antibody, (b) 3.65 mM±0.2 mM L-histidine, (c) 6.35 mM±0.2 mM L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 20 mg/ml±2 mg/ml antibody, (b) 0.57 mg/ml±0.05 mg/ml L-histidine, (c) 1.33 mg/ml±0.13 mg/ml L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 80 mg/ml±8 mg/ml antibody, (b) from 5 mM±1 mM to 15 mM±3 mM histidine buffer, (c) from 0.01%±0.005% to 0.5%±0.25% w/v polysorbate, and (d) from 5%±1% to 20%±4% w/v sucrose, at pH 5.8±0.3.

In some embodiments, the pharmaceutical composition comprises: (a) 80 mg/ml±8 mg/ml antibody, (b) 10 mM±1 mM histidine buffer, (c) 0.1%±0.01% w/v polysorbate, and (d) 10%±1% w/v sucrose, at pH 5.8±0.3.

In some embodiments, the pharmaceutical composition comprises: (a) 80 mg/ml±8 mg/ml antibody, (b) 3.65 mM±0.2 mM L-histidine, (c) 6.35 mM±0.2 mM L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 80 mg/ml±8 mg/ml antibody, (b) 0.57 mg/ml±0.05 mg/ml L-histidine, (c) 1.33 mg/ml±0.13 mg/ml L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 100 mg/ml±10 mg/ml antibody, (b) from 5 mM±1 mM to 15 mM±3 mM histidine buffer, (c) from 0.01%±0.005% to 0.5%±0.25% w/v polysorbate, and (d) from 5%±1% to 20%±4% w/v sucrose, at pH 5.8±0.3.

In some embodiments, the pharmaceutical composition comprises: (a) 100 mg/ml±10 mg/ml antibody, (b) 10 mM±1 mM histidine buffer, (c) 0.1%±0.01% w/v polysorbate, and (d) 10%±1% w/v sucrose, at pH 5.8±0.3.

In some embodiments, the pharmaceutical composition comprises: (a) 100 mg/ml±10 mg/ml antibody, (b) 3.65 mM±0.2 mM L-histidine, (c) 6.35 mM±0.2 mM L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 100 mg/ml±10 mg/ml antibody, (b) 0.57 mg/ml±0.05 mg/ml L-histidine, (c) 1.33 mg/ml±0.13 mg/ml L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 160 mg/ml±16 mg/ml antibody, (b) from 5 mM±1 mM to 15 mM±3 mM histidine buffer, (c) from 0.01%±0.005% to 0.5%±0.25% w/v polysorbate, and (d) from 5%±1% to 20%±4% w/v sucrose, at pH 5.8±0.3.

In some embodiments, the pharmaceutical composition comprises: (a) 160 mg/ml±16 mg/ml antibody, (b) 10 mM±1 mM histidine buffer, (c) 0.1%±0.01% w/v polysorbate, and (d) 10%±1% w/v sucrose, at pH 5.8±0.3.

In some embodiments, the pharmaceutical composition comprises: (a) 160 mg/ml±16 mg/ml antibody, (b) 3.65 mM±0.2 mM L-histidine, (c) 6.35 mM±0.2 mM L-histidine monohydrochloride monohydrate, (d) 0.1%±0.01% w/v polysorbate, and (e) 10%±1% w/v sucrose, at pH 5.8±0.3.

In some embodiments, the pharmaceutical composition comprises: (a) 160 mg/ml±16 mg/ml antibody, (b) 0.57 mg/ml±0.05 mg/ml L-histidine, (c) 1.33 mg/ml±0.13 mg/ml L-histidine monohydrochloride monohydrate, (d) 0.1%±0.01% w/v polysorbate, and (e) 10%±1% w/v sucrose, at pH 5.8±0.3.

In any of these embodiments, the polysorbate may be polysorbate 80.

In various embodiments of the pharmaceutical compositions discussed above or herein, at least 90% of the antibody has native conformation after 1 month of storage at 45° C., as determined by size exclusion-ultra performance liquid chromatography (SE-UPLC). In some cases, at least 93% of the antibody has native conformation after 1 month of storage at 45° C., as determined by SE-UPLC. In some cases, at least 95% of the antibody has native conformation after 1 month of storage at 45° C., as determined by SE-UPLC.

In various embodiments of the pharmaceutical compositions discussed above or herein, at least 95% of the antibody has native conformation after 3 months of storage at 25° C. and 60% relative humidity, as determined by SE-UPLC. In some cases, at least 97% of the antibody has native conformation after 3 months of storage at 25° C. and 60% relative humidity, as determined by SE-UPLC.

In various embodiments of the pharmaceutical compositions discussed above or herein, at least 95% of the antibody has native conformation after 3 months of storage at 5° C., as determined by SE-UPLC. In some cases, at least 98% of the antibody has native conformation after 3 months of storage at 5° C., as determined by SE-UPLC.

In various embodiments of the pharmaceutical compositions discussed above or herein, the formulation contains no more than 6% high molecular weight (HMW) species after 1 month of storage at 45° C., as determined by SE-UPLC. In some cases, the formulation contains no more than 5.6% HMW species after 1 month of storage at 45° C., as determined by SE-UPLC.

In various embodiments of the pharmaceutical compositions discussed above or herein, the formulation contains no more than 2% HMW species after 3 months of storage at 25° C. and 60% relative humidity, as determined by SE-UPLC. In some cases, the formulation contains no more than 1% HMW species after 3 months of storage at 25° C. and 60% relative humidity, as determined by SE-UPLC.

In various embodiments of the pharmaceutical compositions discussed above or herein, the formulation contains no more than 1.5% HMW species after 3 months of storage at 5° C., as determined by SE-UPLC. In some cases, the formulation contains no more than 1% HMW species after 9 months of storage at 5° C., as determined by SE-UPLC.

In various embodiments of any of the pharmaceutical compositions discussed above or herein, the antibody comprises A1-HCDR1, A1-HCDR2 and A1-HCDR3 comprising the amino acid sequences, respectively, of SEQ ID NOs: 7, 8 and 9; A2-HCDR1, A2-HCDR2 and A2-HCDR3 comprising the amino acid sequences, respectively, of SEQ ID NOs: 10, 11 and 12; and LCDR1, LCDR2 and LCDR3 comprising the amino acid sequences, respectively, of SEQ ID NOs: 13, 14 and 15.

In some embodiments, the first antigen-binding domain comprises a HCVR with at least 90% identity to the amino acid sequence of SEQ ID NO: 4 and a LCVR with at least 90% identity to the amino acid sequence of SEQ ID NO: 6, and the second antigen-binding domain comprises a HCVR with at least 90% identity to the amino acid sequence of SEQ ID NO: 5 and a LCVR with at least 90% identity to the amino acid sequence of SEQ ID NO: 6. In some embodiments, the first antigen-binding domain comprises a HCVR with at least 95% identity to the amino acid sequence of SEQ ID NO: 4 and a LCVR with at least 95% identity to the amino acid sequence of SEQ ID NO: 6, and the second antigen-binding domain comprises a HCVR with at least 95% identity to the amino acid sequence of SEQ ID NO: 5 and a LCVR with at least 95% identity to the amino acid sequence of SEQ ID NO: 6. In some embodiments, the first antigen-binding domain comprises a HCVR with at least 99% identity to the amino acid sequence of SEQ ID NO: 4 and a LCVR with at least 99% identity to the amino acid sequence of SEQ ID NO: 6, and the second antigen-binding domain comprises a HCVR with at least 99% identity to the amino acid sequence of SEQ ID NO: 5 and a LCVR with at least 99% identity to the amino acid sequence of SEQ ID NO: 6.

In various embodiments of any of the pharmaceutical compositions discussed above or herein, the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6.

In some embodiments, the antibody comprises a human IgG heavy chain constant region attached, respectively, to the HCVR of each of the first antigen-binding domain and the second antigen-binding domain. In some cases, the heavy chain constant region is of isotype IgG1. In some cases, the heavy chain constant region is of isotype IgG4. In some embodiments, the heavy chain constant region attached to the HCVR of the first antigen-binding domain or the heavy chain constant region attached to the HCVR of the second antigen-binding domain, but not both, contains an amino acid modification that reduces Protein A binding relative to a heavy chain of the same isotype without the modification. In some cases, the modification comprises a H435R substitution (EU numbering) in a heavy chain of isotype IgG1 or IgG4. In some cases, the modification comprises a H435R substitution and a Y436F substitution (EU numbering) in a heavy chain of isotype IgG1 or IgG4.

In some embodiments, the antibody comprises a chimeric hinge. For example, the chimeric hinge comprises, in an embodiment, a first amino acid sequence, or an “upper hinge” sequence, derived from a human IgG1 hinge region or human IgG4 hinge region, and a second amino acid sequence, or a “lower hinge” sequence, derived from a human IgG2 hinge region. In certain embodiments, the first or “upper hinge” sequence comprises amino acid residues from positions 216 to 227 according to EU numbering. In some embodiments, the second or “lower hinge” sequence comprises amino acid residues from positions 228 to 236 according to EU numbering. In some cases in which the antibody heavy chain constant region is of isotype IgG4, the chimeric hinge comprises an upper hinge sequence from human IgG4 (positions 216 to 227 according to EU numbering), and a lower hinge sequence from human IgG2 (positions 228 to 236 according to EU numbering). In some cases in which the antibody heavy chain constant region is of isotype IgG1, the chimeric hinge comprises an upper hinge sequence from human IgG1 (positions 216 to 227 according to EU numbering), and a lower hinge sequence from human IgG2 (positions 228 to 236 according to EU numbering). In some embodiments in which the heavy chain constant region is of isotype IgG1, and the antibody comprises a chimeric hinge, the CH2 domain of the otherwise IgG1 heavy chain constant region is of isotype IgG4. Unless otherwise indicated, references to an IgG1 or IgG4 heavy chain constant region include heavy chain constant regions comprising a chimeric hinge (e.g., an IgG1 or IgG4 upper hinge sequence, respectively, and an IgG2 lower hinge sequence). For example, reference to an IgG1 heavy chain constant region includes an IgG1 heavy chain constant region that comprises an IgG2 lower hinge sequence, and reference to an IgG4 heavy chain constant region includes an IgG4 heavy chain constant region that comprises an IgG2 lower hinge sequence.

In some embodiments, the bispecific antibody comprises a heavy chain constant region comprising an amino acid sequence selected from the group consisting of SEQ ID NOs: 16, 17, 18 and 19. In some embodiments, the bispecific antibody comprises a heavy chain constant region comprising the amino acid sequence of SEQ ID NO: 16 and a heavy chain constant region comprising the amino acid sequence of SEQ ID NO: 17. In some embodiments, the bispecific antibody comprises a heavy chain constant region comprising the amino acid sequence of SEQ ID NO: 18 and a heavy chain constant region comprising the amino acid sequence of SEQ ID NO: 19.

In various embodiments of any of the pharmaceutical compositions discussed above or herein, the antibody comprises a first heavy chain containing the HCVR of the first antigen-binding domain and a second heavy chain containing the HCVR of the second antigen-binding domain, wherein the first heavy chain comprises residues 1-452 of the amino acid sequence of SEQ ID NO: 1 and the second heavy chain comprises residues 1-448 of the amino acid sequence of SEQ ID NO: 2. In some embodiments, the antibody further comprises a common light chain containing the LCVR of the first and second antigen-binding domains, wherein the common light chain comprises the amino acid sequence of SEQ ID NO: 3.

In various embodiments of any of the pharmaceutical compositions discussed above or herein, the antibody comprises a first heavy chain containing the HCVR of the first antigen-binding domain and a second heavy chain containing the HCVR of the second antigen-binding domain, wherein the first heavy chain comprises the amino acid sequence of SEQ ID NO: 1 and the second heavy chain comprises the amino acid sequence of SEQ ID NO: 2. In some embodiments, the antibody further comprises a common light chain containing the LCVR of the first and second antigen-binding domains, wherein the common light chain comprises the amino acid sequence of SEQ ID NO: 3.

In one aspect, the present invention provides a pharmaceutical formulation comprising: (a) 2 mg/ml±0.2 mg/ml of a bispecific antibody comprising a first antigen-binding domain that binds specifically to human CD20 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6; (b) 10 mM±1 mM histidine buffer, pH 5.8±0.2, (c) 0.1%±0.01% w/v polysorbate 80, and (d) 10%±1% w/v sucrose.

In some embodiments, the pharmaceutical formulation consists of: (a) 2 mg/ml±0.2 mg/ml of the antibody, (b) 3.65 mM±0.2 mM L-histidine (c) 6.35 mM±0.2 mM L-histidine monohydrochloride monohydrate, and (d) 0.1%±0.01% w/v polysorbate 80, in water at pH 5.8±0.2. In some embodiments, the pharmaceutical formulation consists of: (a) 2 mg/ml±0.2 mg/ml of the antibody, (b) 0.57 mg/ml±0.1 mg/ml L-histidine (c) 1.33 mg/ml±0.1 mg/ml L-histidine monohydrochloride monohydrate, (d) 1 mg/ml±0.1 mg/ml polysorbate 80, and (e) 100 mg/ml±10 mg/ml sucrose, in water at pH 5.8±0.2.

In one aspect, the present invention provides a pharmaceutical formulation comprising: (a) 20 mg/ml±2 mg/ml of a bispecific antibody comprising a first antigen-binding domain that binds specifically to human CD20 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6; (b) 10 mM±1 mM histidine buffer, pH 5.8±0.2, (c) 0.1%±0.01% w/v polysorbate 80, and (d) 10%±1% w/v sucrose.

In some embodiments, the pharmaceutical formulation consists of: (a) 20 mg/ml±2 mg/ml of the antibody, (b) 3.65 mM±0.2 mM L-histidine (c) 6.35 mM±0.2 mM L-histidine monohydrochloride monohydrate, and (d) 0.1%±0.01% w/v polysorbate 80, in water at pH 5.8±0.2. In some embodiments, the pharmaceutical formulation consists of: (a) 20 mg/ml±2 mg/ml of the antibody, (b) 0.57 mg/ml±0.1 mg/ml L-histidine (c) 1.33 mg/ml±0.1 mg/ml L-histidine monohydrochloride monohydrate, (d) 1 mg/ml±0.1 mg/ml polysorbate 80, and (e) 100 mg/ml±10 mg/ml sucrose, in water at pH 5.8±0.2.

In one aspect, the present invention provides a pharmaceutical formulation comprising: (a) 80 mg/ml±8 mg/ml of a bispecific antibody comprising a first antigen-binding domain that binds specifically to human CD20 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6; (b) 10 mM±1 mM histidine buffer, pH 5.8±0.2, (c) 0.1%±0.01% w/v polysorbate 80, and (d) 10%±1% w/v sucrose.

In some embodiments, the pharmaceutical formulation consists of: (a) 80 mg/ml±8 mg/ml of the antibody, (b) 3.65 mM±0.2 mM L-histidine (c) 6.35 mM±0.2 mM L-histidine monohydrochloride monohydrate, and (d) 0.1%±0.01% w/v polysorbate 80, in water at pH 5.8±0.2. In some embodiments, the pharmaceutical formulation consists of: (a) 80 mg/ml±8 mg/ml of the antibody, (b) 0.57 mg/ml±0.1 mg/ml L-histidine (c) 1.33 mg/ml±0.1 mg/ml L-histidine monohydrochloride monohydrate, (d) 1 mg/ml±0.1 mg/ml polysorbate 80, and (e) 100 mg/ml±10 mg/ml sucrose, in water at pH 5.8±0.2.

In one aspect, the present invention provides a pharmaceutical formulation comprising: (a) 100 mg/ml±10 mg/ml of a bispecific antibody comprising a first antigen-binding domain that binds specifically to human CD20 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6; (b) 10 mM±1 mM histidine buffer, pH 5.8±0.2, (c) 0.1%±0.01% w/v polysorbate 80, and (d) 10%±1% w/v sucrose.

In some embodiments, the pharmaceutical formulation consists of: (a) 100 mg/ml±10 mg/ml of the antibody, (b) 3.65 mM±0.2 mM L-histidine (c) 6.35 mM±0.2 mM L-histidine monohydrochloride monohydrate, and (d) 0.1%±0.01% w/v polysorbate 80, in water at pH 5.8±0.2. In some embodiments, the pharmaceutical formulation consists of: (a) 100 mg/ml±10 mg/ml of the antibody, (b) 0.57 mg/ml±0.1 mg/ml L-histidine (c) 1.33 mg/ml±0.1 mg/ml L-histidine monohydrochloride monohydrate, (d) 1 mg/ml±0.1 mg/ml polysorbate 80, and (e) 100 mg/ml±10 mg/ml sucrose, in water at pH 5.8±0.2.

In some embodiments, the pharmaceutical formulation comprises: (a) from 1 to 200 mg/ml of the antibody (as discussed above or herein); (b) a buffer comprising acetate or phosphate; (c) an organic co-solvent comprising polysorbate; and (d) a stabilizer comprising a sugar; wherein the formulation has a pH of 5.8±0.3. In some embodiments, the pharmaceutical formulation comprises: (a) from 1 to 200 mg/ml of the antibody (e.g., 2 mg/ml, 20 mg/ml, 80 mg/ml or 100 mg/ml), (b) from 1-50 mM acetate or phosphate buffer, and (d) 0.1%±0.01% w/v polysorbate 80, in water at pH 5.8±0.2.

In one aspect, the present invention provides a pharmaceutical composition, wherein the composition comprises the pharmaceutical formulation as discussed above or herein, and the composition is contained in a container. In some embodiments, the container is a vial. In some cases, the vial is a 2 ml, 5 ml, 10 ml or 20 ml Type 1 clear glass vial. In some embodiments, the container is a syringe. In some cases, the syringe is low-tungsten glass. In some embodiments, the container is a prefilled syringe. In some embodiments, the composition is contained in an autoinjector.

In one aspect, the present invention provides a kit comprising (i) a container containing a composition comprising the pharmaceutical formulation as discussed above or herein, and instructions for use of the composition. In some embodiments, the container is a glass vial. In some embodiments, the container is a prefilled syringe. In some embodiments, the container is an autoinjector. In some cases, the instructions recite subcutaneous administration of the composition. In some cases, the instructions recite intravenous administration of the composition.

In one embodiment, the present invention provides a container, optionally wherein the container is a glass vial, containing 2 mg antibody in a stable formulation, wherein the formulation comprises (a) 2 mg/ml±0.2 mg/ml of the antibody, (b) 0.57 mg/ml±0.1 mg/ml L-histidine (c) 1.33 mg/ml±0.1 mg/ml L-histidine monohydrochloride monohydrate, (d) 1 mg/ml±0.1 mg/ml polysorbate 80, and (e) 100 mg/ml±10 mg/ml sucrose, in water at pH 5.8±0.2. In one embodiment, the present invention provides a container, optionally wherein the container is a glass vial, containing 20 mg, 80 mg or 160 mg antibody in a stable formulation, wherein the formulation comprises (a) 20 mg/ml±2 mg/ml of the antibody, (b) 0.57 mg/ml±0.1 mg/ml L-histidine (c) 1.33 mg/ml±0.1 mg/ml L-histidine monohydrochloride monohydrate, (d) 1 mg/ml±0.1 mg/ml polysorbate 80, and (e) 100 mg/ml±10 mg/ml sucrose, in water at pH 5.8±0.2. In one embodiment, the present invention provides a container, optionally wherein the container is a glass vial, containing 100 mg, 200 mg or 400 mg antibody in a stable formulation, wherein the formulation comprises (a) 100 mg/ml±10 mg/ml of the antibody, (b) 0.57 mg/ml±0.1 mg/ml L-histidine (c) 1.33 mg/ml±0.1 mg/ml L-histidine monohydrochloride monohydrate, (d) 1 mg/ml±0.1 mg/ml polysorbate 80, and (e) 100 mg/ml±10 mg/ml sucrose, in water at pH 5.8±0.2.

In one aspect, the present invention provides a unit dosage form comprising the pharmaceutical formulation as discussed above or herein, wherein the antibody is present in an amount of from 0.1 mg to 500 mg. In some cases, the antibody is present in an amount of from 2 to 2.5 mg. In some cases, the antibody is present in an amount of from 10 to 11 mg. In some cases, the antibody is present in an amount of from 20 to 25 mg. In some cases, the antibody is present in an amount of from 80 to 90 mg. In some cases, the antibody is present in an amount of from 100 to 125 mg. In some cases, the antibody is present in an amount of from 160 to 180 mg. In some cases, the antibody is present in an amount of from 190 to 210 mg. In some cases, the antibody is present in an amount of from 320 to 360 mg. In some embodiments, the unit dosage form is a glass vial. In some embodiments, the unit dosage form is a prefilled syringe. In some embodiments, the unit dosage form is an autoinjector.

In any of the pharmaceutical compositions, kits, or unit dosage forms discussed above or herein, the container containing the pharmaceutical formulation may comprise a headspace comprising a gas in which the gas comprises less than 5% oxygen by volume. In some cases, the gas comprises less than 1% oxygen by volume. In some cases, the gas comprises no more than 0.1% oxygen by volume.

In one aspect, the present invention provides a container containing a pharmaceutical composition, wherein the composition comprises the pharmaceutical formulation as discussed above or herein. In some cases, the container is a syringe. In some cases, the container is a prefilled syringe. In some cases, the container is an autoinjector. In some cases, the container is a glass vial.

In various embodiments, any of the features or components of embodiments discussed above or herein may be combined, and such combinations are encompassed within the scope of the present disclosure. Any specific value discussed above or herein may be combined with another related value discussed above or herein to recite a range with the values representing the upper and lower ends of the range, and such ranges and all values falling within such ranges are encompassed within the scope of the present disclosure. Each of the values discussed above or herein may be expressed with a variation of 1%, 5%, 10% or 20%. For example, a concentration of 10 mM may be expressed as 10 mM±0.1 mM (1% variation), 10 mM±0.5 mM (5% variation), 10 mM±1 mM (10% variation) or 10 mM±2 mM (20% variation).

Other embodiments will become apparent from a review of the ensuing detailed description.

### DETAILED DESCRIPTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. As used herein, the term “about,” when used in reference to a particular recited numerical value or range of values, means that the value may vary from the recited value by no more than 1%. For example, as used herein, the expression “about 100” includes 99 and 101 and all values in between (e.g., 99.1, 99.2, 99.3, 99.4, etc.).

Although any methods and materials similar or equivalent to those described herein can be used in the practice or testing of the present invention, exemplary methods and materials are now described. All patents, applications and non-patent publications mentioned in this specification are incorporated herein by reference in their entireties.

### Pharmaceutical Formulations

As used herein, the expression “pharmaceutical formulation” means a combination of at least one active ingredient (e.g., a bispecific anti-CD20×anti-CD3 antibody, which is capable of exerting a biological effect in a human or non-human animal), and at least one inactive ingredient which, when combined with the active ingredient and/or one or more additional inactive ingredients, is suitable for therapeutic administration to a human or non-human animal. The term “formulation,” as used herein, means “pharmaceutical formulation” unless specifically indicated otherwise. The present invention provides pharmaceutical formulations comprising at least one therapeutic polypeptide. According to certain embodiments of the present invention, the therapeutic polypeptide is a bispecific antibody that binds specifically to human CD20 and human CD3 or an antigen-binding fragment thereof. More specifically, the present invention includes pharmaceutical formulations that comprise: (i) a human bispecific antibody that specifically binds to human CD20 and human CD3; (ii) a buffer comprising histidine; (iii) an organic co-solvent comprising polysorbate; and (iv) a stabilizer comprising a sugar. Additional components may be included in the formulations of the present invention if such components do not significantly interfere with the stability of the formulation. Specific exemplary components and formulations included within the present invention are described in detail below.

The pharmaceutical formulations of the present invention may, in certain embodiments, be fluid formulations. As used herein, the expression “fluid formulation” means a mixture of at least two components that exists predominantly in the fluid state at about 2° C. to about 45° C. Fluid formulations include, inter alia, liquid formulations. Fluid formulations may be of low, moderate or high viscosity depending on their particular constituents.

Bispecific Antibodies that Specifically Bind Human CD20 and Human CD3

The pharmaceutical formulations of the present invention may comprise a human bispecific antibody, or an antigen-binding fragment thereof, that binds specifically to human CD20 and human CD3.

The term “antibody,” as used herein, which includes a “bispecific antibody,” is generally intended to refer to immunoglobulin molecules comprising four polypeptide chains, two heavy (H) chains and two light (L) chains inter-connected by disulfide bonds, as well as multimers thereof (e.g., IgM); however, immunoglobulin molecules consisting of only heavy chains (i.e., lacking light chains) are also encompassed within the definition of the term “antibody.” Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementary determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

In certain embodiments of the invention, the anti-CD20×anti-CD3 bispecific antibodies of the invention are human antibodies. The term “human antibody,” as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences. In various embodiments, the anti-CD20×anti-CD3 bispecific antibody is a human IgG antibody. In various embodiments, the anti-CD20×anti-CD3 bispecific antibody is a human antibody of isotype IgG1, IgG2, IgG3 or IgG4, or mixed isotype. In some embodiments, the anti-CD20×anti-CD3 bispecific antibody is a human IgG1 antibody (i.e., the antibody comprises a human IgG1 heavy chain constant region attached, respectively, to the HCVR of each of the first antigen-binding domain and the second antigen-binding domain). In some embodiments, the anti-CD20×anti-CD3 bispecific antibody is a human IgG4 antibody (i.e., the antibody comprises a human IgG4 heavy chain constant region attached, respectively, to the HCVR of each of the first antigen-binding domain and the second antigen-binding domain. In any of the embodiments discussed above or herein, the anti-CD20×anti-CD3 bispecific antibody may comprise a human kappa light chain. In any of the embodiments discussed above or herein, the anti-CD20×anti-CD3 bispecific antibody may comprise a human lambda light chain.

In any embodiments, the bispecific antibody may include a modification in one or both heavy chains to facilitate purification of the bispecific antibody (i.e., the heterodimer) from homodimeric impurities. In some embodiments, the bispecific antibodies include first and second heavy chains (i.e., the heavy chain of the anti-CD20 binding arm, and the heavy chain of the anti-CD3 binding arm) that are identical (e.g., both of isotype IgG1 or IgG4) except for a modification in the CH3 domain of one or the other heavy chain that reduces binding of the bispecific antibody to Protein A as compared to an antibody lacking the modification. In some cases, the CH3 domain of the first heavy chain (e.g., of the anti-CD20 binding arm) binds Protein A and the CH3 domain of the second heavy chain (e.g., of the anti-CD3 binding arm) contains a mutation that reduces or abolishes Protein A binding. In some cases, the mutation is a H435R modification (by EU numbering; H95R by IMGT exon numbering). In some cases, the mutation is a H435R modification (by EU numbering; H95R by IMGT exon numbering) and a Y436F modification (by EU numbering; Y96F by IMGT). Further modifications that may be found within the second CH3 domain include: D356E, L358M, N384S, K392N, V397M, and V422I by EU (D16E, L18M, N44S, K52N, V57M, and V82I by IMGT) in the case of IgG1 CH3 domains; and Q355R, N384S, K392N, V397M, R409K, E419Q, and V422I by EU (Q15R, N44S, K52N, V57M, R69K, E79Q, and V82I by IMGT) in the case of IgG4 CH3 domains.

In any embodiments, the bispecific antibody may include a chimeric hinge. The term “chimeric hinge” is intended to include a chimeric protein comprising a first amino acid sequence derived from the hinge region of one Ig molecule and a second amino acid sequence derived from the hinge region of a different class or subclass of Ig molecule. For example, the chimeric hinge comprises, in an embodiment, a first amino acid sequence, or an “upper hinge” sequence, derived from a human IgG1 hinge region or human IgG4 hinge region, and a second amino acid sequence, or a “lower hinge” sequence, derived from a human IgG2 hinge region. In certain embodiments, the first or “upper hinge” sequence comprises amino acid residues from positions 216 to 227 according to EU numbering. In some embodiments, the second or “lower hinge” sequence comprises amino acid residues from positions 228 to 236 according to EU numbering.

The antibodies of the invention may, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell, antibodies isolated from a recombinant, combinatorial human antibody library, antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The terms “antigen-binding portion” or “antigen-binding fragment” of an antibody (or simply “antibody portion” or “antibody fragment”), as used herein, refer to one or more fragments of an antibody that retain the ability to specifically bind to human CD20 or human CD3.

An “isolated antibody,” as used herein, is intended to refer to an antibody that is substantially free of other antibodies having different antigenic specificities (e.g., an isolated bispecific antibody that specifically binds human CD20 and human CD3 is substantially free of antibodies that specifically bind antigens other than human CD20 and human CD3).

The term “specifically binds,” or the like, means that an antibody or antigen-binding fragment thereof forms a complex with an antigen that is relatively stable under physiologic conditions. Specific binding can be characterized by a dissociation constant of at least about 1×10⁻⁶ M or greater. Methods for determining whether two molecules specifically bind are well known in the art and include, for example, equilibrium dialysis, surface plasmon resonance, and the like. An isolated antibody that specifically binds human CD20 and human CD3 may, however, have cross-reactivity to other antigens, such as CD20 or CD3 molecules from other species (orthologs). In the context of the present invention, multispecific (e.g., bispecific) antibodies that bind to human CD20 and human CD3 as well as one or more additional antigens are deemed to “specifically bind” human CD20 and human CD3. Moreover, an isolated antibody may be substantially free of other cellular material and/or chemicals.

Exemplary anti-CD20×anti-CD3 bispecific antibodies that may be included in the pharmaceutical formulations of the present invention are set forth in WO 2014/047231, the disclosure of which is incorporated by reference in its entirety.

According to certain embodiments of the present invention, the anti-CD20×anti-CD3 bispecific antibody, or antigen-binding fragment thereof, comprises a first antigen-binding domain that specifically binds human CD20 and a second antigen-binding domain that specifically binds human CD3, in which the first antigen-binding domain comprises heavy chain complementarity determining regions (CDRs) A1-HCDR1, A1-HCDR2, and A1-HCDR3, respectively, comprising the amino acid sequences of SEQ ID NOs: 7, 8, and 9, and the second antigen-binding domain comprises heavy chain CDRs A2-HCDR1, A2-HCDR2, and A2-HCDR3, respectively, comprising the amino acid sequences of SEQ ID NOs: 10, 11, and 12. According to certain embodiments of the present invention, the anti-CD20×anti-CD3 bispecific antibody, or antigen-binding fragment thereof, comprises common (to both the first and second antigen-binding domains) light chain complementarity determining regions LCDR1-LCDR2-LCDR3, respectively, comprising the amino acid sequences of SEQ ID NOs: 13, 14, and 15.

In certain embodiments, the anti-CD20×anti-CD3 bispecific antibody, or antigen-binding fragment thereof, comprises a first antigen-binding domain that specifically binds human CD20 and a second antigen-binding domain that specifically binds human CD3, in which the first antigen-binding domain comprises a heavy chain variable region (HCVR) comprising the amino acid sequence of SEQ ID NO: 4, and the second antigen-binding domain comprises a HCVR comprising the amino acid sequence of SEQ ID NO: 5. In certain embodiments, the anti-CD20×anti-CD3 bispecific antibody, or antigen-binding fragment thereof, comprises a common light chain variable region (LCVR) comprising the amino acid sequence of SEQ ID NO: 6. In certain embodiments, the anti-CD20×anti-CD3 bispecific antibody, or antigen-binding fragment thereof, comprises a first antigen-binding domain comprising a HCVR/LCVR amino acid sequence pair comprising the amino acid sequences of SEQ ID NOs: 4/6, and a second antigen-binding domain comprising a HCVR/LCVR amino acid sequence pair comprising the amino acid sequences of SEQ ID NOs: 5/6. In some embodiments, the anti-CD20×anti-CD3 bispecific antibody comprises the HCVR/LCVR sequence pairs noted above, and a human IgG1 heavy chain constant region. In some embodiments, the anti-CD20×anti-CD3 bispecific antibody comprises the HCVR/LCVR sequence pairs noted above, and a human IgG4 heavy chain constant region. In some embodiments, the anti-CD20×anti-CD3 bispecific antibody comprises the HCVR/LCVR sequence pairs noted above, and a human IgG heavy chain constant region. In some embodiments, the anti-CD20×anti-CD3 bispecific antibody comprises the HCVR/LCVR sequence pairs noted above, and a human IgG1 or IgG4 heavy chain constant region. In some embodiments, the anti-CD20×anti-CD3 bispecific antibody comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3. An exemplary anti-CD20×anti-CD3 bispecific antibody with a first antigen-binding domain that specifically binds human CD20 and comprises a HCVR comprising the amino acid sequence of SEQ ID NO: 4 and a LCVR comprising the amino acid sequence of SEQ ID NO: 6, and a second antigen-binding domain that specifically binds human CD3 and comprises a HCVR comprising the amino acid sequence of SEQ ID NO: 5 and a LCVR comprising the amino acid sequence of SEQ ID NO: 6 is referred to herein as REGN1979. In some embodiments, the bispecific antibody has a first heavy chain (including the HCVR that specifically binds human CD20) comprising the amino acid sequence of SEQ ID NO: 1, a second heavy chain (including the HCVR that specifically binds human CD3) comprising the amino acid sequence of SEQ ID NO: 2, and a common light chain comprising the amino acid sequence of SEQ ID NO: 3. In some cases, the mature form of the antibody may not include the C-terminal lysine residues of SEQ ID NOs: 1 and 2. Thus, in some cases the anti-CD20 binding arm of the antibody comprises a heavy chain comprising residues 1-452 of SEQ ID NO: 1, and the anti-CD3 binding arm of the antibody comprises a heavy chain comprising residues 1-448 of SEQ ID NO: 2.

The amount of antibody, or antigen-binding fragment thereof, contained within the pharmaceutical formulations of the present invention may vary depending on the specific properties desired of the formulations, as well as the particular circumstances and purposes for which the formulations are intended to be used. In certain embodiments, the pharmaceutical formulations may contain about 0.1 mg/mL to about 500 mg/mL of antibody; about 0.5 mg/mL to about 400 mg/mL of antibody; about 1 mg/mL to about 200 mg/mL of antibody; about 2 mg/mL to about 100 mg/mL; about 1 mg/mL to about 5 mg/mL of antibody; about 10 mg/mL to about 30 mg/mL of antibody; about 75 mg/mL to about 125 mg/mL; about 5 mg/mL to about 50 mg/mL; or about 2 mg/mL to about 160 mg/mL of antibody. For example, the formulations of the present invention may by liquid formulations that comprise about 0.5 mg/mL; about 1 mg/mL; about 2 mg/mL; about 3 mg/mL; about 4 mg/mL; about 5 mg/mL; about 6 mg/mL; about 7 mg/mL, about 8 mg/mL; about 9 mg/mL; about 10 mg/mL; about 11 mg/mL; about 12 mg/mL; about 13 mg/mL; about 14 mg/mL; about 15 mg/mL; about 16 mg/mL; about 17 mg/mL; about 18 mg/mL; about 19 mg/mL; about 20 mg/mL; about 21 mg/mL; about 22 mg/mL; about 23 mg/mL; about 24 mg/mL; about 25 mg/mL; about 26 mg/mL; about 27 mg/mL; about 28 mg/mL; about 29 mg/mL; about 30 mg/mL; about 35 mg/mL; about 40 mg/mL; about 45 mg/mL; about 50 mg/mL; about 55 mg/mL; about 60 mg/mL; about 65 mg/mL; about 70 mg/mL; about 75 mg/mL; about 80 mg/mL; about 85 mg/mL; about 90 mg/mL; about 95 mg/mL; about 96 mg/mL; about 97 mg/mL; about 98 mg/mL; about 99 mg/mL; about 100 mg/mL; about 101 mg/mL; about 102 mg/mL; about 103 mg/mL; about 104 mg/mL; about 105 mg/mL; about 110 mg/mL; about 115 mg/mL; about 120 mg/mL; about 125 mg/mL; about 130 mg/mL; about 135 mg/mL; about 140 mg/mL; about 145 mg/mL; about 150 mg/mL; about 155 mg/mL; about 160 mg/mL; about 165 mg/mL; about 170 mg/mL; about 175 mg/mL; about 180 mg/mL; about 185 mg/mL; about 190 mg/mL; about 195 mg/mL; or about 200 mg/mL of an antibody or an antigen-binding fragment thereof, that binds specifically to human CD20 and human CD3. In certain embodiments, the pharmaceutical formulations are liquid formulations that may contain 1±0.1 mg/mL to 200±20 mg/mL of antibody; 2±0.2 mg/mL to 100±10 mg/mL of antibody; 1±0.5 mg/mL to 30±5 mg/mL of antibody; 10±1 mg/mL to 30±3 mg/mL of antibody; 1±0.1 mg/mL to 3±0.3 mg/mL of antibody; 15±1.5 mg/mL to 25±2.5 mg/mL of antibody; 90±5 mg/mL to 110±5 mg/mL of antibody; or 150±7.5 mg/mL to 170±7.5 mg/mL of antibody. In some embodiments, the pharmaceutical formulations contain 2±0.2 mg/mL of antibody. In some embodiments, the pharmaceutical formulations contain 20±2 mg/mL of antibody. In some embodiments, the pharmaceutical formulations contain 80±8 mg/ml of antibody. In some embodiments, the pharmaceutical formulations contain 100±10 mg/ml of antibody.

### Bioequivalents

The present invention encompasses antibodies having amino acid sequences that vary from those of the exemplary molecules disclosed herein but that retain the ability to bind human CD20 and human CD3. Such variant molecules may comprise one or more additions, deletions, or substitutions of amino acids when compared to parent sequence, but exhibit biological activity that is essentially equivalent to that of the antibodies discussed herein.

The present invention includes antigen-binding molecules that are bioequivalent to any of the exemplary antibodies set forth herein. Two antibodies are considered bioequivalent if, for example, they are pharmaceutical equivalents or pharmaceutical alternatives whose rate and extent of absorption do not show a significant difference when administered at the same molar dose under similar experimental conditions, either single does or multiple dose. Some antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antibodies are bioequivalent if there are no clinically meaningful differences in their safety, purity, and potency.

In one embodiment, two antibodies are bioequivalent if a patient can be switched one or more times between the reference product and the biological product without an expected increase in the risk of adverse effects, including a clinically significant change in immunogenicity, or diminished effectiveness, as compared to continued therapy without such switching.

Bioequivalence may be demonstrated by in vivo and in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antigen-binding protein.

### Formulation Excipients and pH

The pharmaceutical formulations of the present invention comprise one or more excipients. The term “excipient,” as used herein, means any non-therapeutic agent added to the formulation to provide a desired consistency, viscosity or stabilizing effect.

In certain embodiments, the pharmaceutical formulations of the present invention have a viscosity of less than 15 cP, which is advantageous for delivering the compositions from a prefilled syringe or an autoinjector. In some cases, the pharmaceutical formulations have a viscosity of less than 14 cP, less than 13 cP, less than 12 cP, less than 11 cP, less than 10 cP, less than 9 cP, less than 8 cP, less than 7 cP, less than 6 cP, less than 5 cP, less than 4 cP, less than 3 cP, less than 2 cP, or less than 1.5 cP at 20° C. when measured using a capillary viscometer (e.g., as discussed in Example 3). In some embodiments, the pharmaceutical formulations have a viscosity of less than 15 cP at antibody concentrations of up to 150 mg/ml at 20° C. when measured using a capillary viscometer (e.g., as discussed in Example 3). In some embodiments, the pharmaceutical formulations have a viscosity of less than 5 cP at antibody concentrations of up to 100 mg/ml at 20° C. when measured using a capillary viscometer (e.g., as discussed in Example 3). In some embodiments, the pharmaceutical formulations have a viscosity of less than 2 cP at antibody concentrations of up to 20 mg/ml at 20° C. when measured using a capillary viscometer (e.g., as discussed in Example 3).

In certain embodiments, the pharmaceutical formulations of the present invention comprise one or more carbohydrates, e.g., one or more sugars. The sugar can be a reducing sugar or a non-reducing sugar. “Reducing sugars” include, e.g., sugars with a ketone or aldehyde group and contain a reactive hemiacetal group, which allows the sugar to act as a reducing agent. Specific examples of reducing sugars include fructose, glucose, glyceraldehyde, lactose, arabinose, mannose, xylose, ribose, rhamnose, galactose and maltose. Non-reducing sugars can comprise an anomeric carbon that is an acetal and is not substantially reactive with amino acids or polypeptides to initiate a Maillard reaction. Specific examples of non-reducing sugars include sucrose, trehalose, sorbose, sucralose, melezitose and raffinose. Sugar acids include, for example, saccharic acids, gluconate and other polyhydroxy sugars and salts thereof. In some embodiments, the sugar is sucrose. In some cases, the sugar (e.g., sucrose) acts as a thermal stabilizer for the anti-CD20×anti-CD3 bispecific antibody.

The amount of sugar (e.g., sucrose) contained within the pharmaceutical formulations of the present invention will vary depending on the specific circumstances and intended purposes for which the formulations are used. In certain embodiments, the formulations may contain about 0.1% to about 20% sugar; about 0.5% to about 20% sugar; about 1% to about 20% sugar; about 2% to about 15% sugar; about 5% to about 15% sugar; about 7.5% to about 12.5% sugar; or about 9% to about 11% sugar. For example, the pharmaceutical formulations of the present invention may comprise about 0.5%; about 1.0%; about 1.5%; about 2.0%; about 2.5%; about 3.0%; about 3.5%; about 4.0%; about 4.5%; about 5.0%; about 5.5%; about 6.0%; about 6.5%; about 7.0%; about 7.5%; about 8.0%; about 8.5%; about 9.0%; about 9.5%; about 10.0%; about 10.5%; about 11.0%; about 11.5%; about 12.0%; about 12.5%; about 13.0%; about 13.5%; about 14.0%; about 14.5%; about 15%; or about 20% sugar (e.g., sucrose). In some embodiments, the formulations contain about 10% sugar (e.g., sucrose). Each of the percentages noted above corresponds to a percent weight/volume (w/v). In some cases, the formulations contain from 5%±1% to 20%±4% w/v sucrose. In some cases, the formulations contain from 8%±0.5% to 12%±0.5% w/v sucrose. In some cases, the formulations contain 10%±1% w/v sucrose.

The pharmaceutical formulations of the present invention may also comprise one or more organic co-solvents (or interfacial stabilizer) in a type and in an amount that stabilizes the anti-CD20×anti-CD3 bispecific antibody under conditions of rough handling or agitation, such as, e.g., orbital shaking. In some embodiments, the organic co-solvent is a surfactant. As used herein, the term “surfactant” means a substance which reduces the surface tension of a fluid in which it is dissolved and/or reduces the interfacial tension between oil and water. Surfactants can be ionic or non-ionic. Exemplary non-ionic surfactants that can be included in the formulations of the present invention include, e.g., alkyl poly(ethylene oxide), alkyl polyglucosides (e.g., octyl glucoside and decyl maltoside), fatty alcohols such as cetyl alcohol and oleyl alcohol, cocamide MEA, cocamide DEA, and cocamide TEA. Specific non-ionic surfactants that can be included in the formulations of the present invention include, e.g., polysorbates such as polysorbate 20, polysorbate 28, polysorbate 40, polysorbate 60, polysorbate 65, polysorbate 80, polysorbate 81, and polysorbate 85; poloxamers such as poloxamer 188 (also known as Pluronic F68), poloxamer 407; polyethylene-polypropylene glycol; or polyethylene glycol (PEG). Polysorbate 20 is also known as TWEEN 20, sorbitan monolaurate and polyoxyethylenesorbitan monolaurate. In some embodiments, the surfactant is polysorbate 80.

The amount of surfactant contained within the pharmaceutical formulations of the present invention may vary depending on the specific properties desired of the formulations, as well as the particular circumstances and purposes for which the formulations are intended to be used. In certain embodiments, the formulations may contain about 0.01% to about 1% surfactant; about 0.01% to about 0.5% surfactant; about 0.05% to about 0.15%; about 0.08% to about 0.12% surfactant; or about 0.09% to about 0.11% surfactant. For example, the formulations of the present invention may comprise about 0.01%; about 0.02%; about 0.03%; about 0.04%; about 0.05%; about 0.06%; about 0.07%; about 0.08%; about 0.09%; about 0.10%; about 0.11%; about 0.12%; about 0.13%; about 0.14%; about 0.15%; about 0.16%; about 0.17%; about 0.18%; about 0.19%; about 0.20%; about 0.21%; about 0.22%; about 0.23%; about 0.24%; about 0.25%; about 0.26%; about 0.27%; about 0.28%; about 0.29%; or about 0.30% surfactant (e.g., polysorbate 80). In some embodiments, the formulations contain about 0.1% surfactant (e.g., polysorbate 80). Each of the percentages noted above corresponds to a percent weight/volume (w/v). In some cases, the formulations contain from 0.01%±0.005% to 0.5%±0.25% w/v polysorbate 80. In some cases, the formulations contain 0.1%±0.05% w/v polysorbate 80. In some cases, the formulations contain 0.1%±0.01% w/v polysorbate 80.

The pharmaceutical formulations of the present invention may also comprise a buffer or buffer system, which serves to maintain a stable pH and to help stabilize the anti-CD20×anti-CD3 bispecific antibody. In some embodiments, the buffer or buffer system comprises at least one buffer that has a buffering range that overlaps fully or in part the range of pH 5.5 to 6.1. In certain embodiments, the buffer comprises a histidine buffer. In certain embodiments, the buffer (e.g., histidine) is present at a concentration of from about 1 mM to about 40 mM, about 1 mM to about 30 mM, about 1 mM to about 20 mM; about 3 mM to about 18 mM; about 5 mM to about 15 mM; or about 8 mM to about 12 mM. In some embodiments, the buffer (e.g., histidine) is present at a concentration of about 1 mM; about 2 mM; about 3 mM; about 4 mM; about 5 mM; about 6 mM; about 7 mM; about 8 mM; about 9 mM; about 10 mM; about 11 mM; about 12 mM; about 13 mM; about 14 mM; about 15 mM; about 16 mM; about 17 mM; about 18 mM; about 19 mM; or about 20 mM. In some cases, the formulations contain a histidine buffer at a concentration of from 5 mM±1 mM to 15 mM±3 mM. In some cases, the formulations contain a histidine buffer at a concentration of 10 mM±1 mM. In some embodiments, the histidine buffer comprises L-histidine and L-histidine monohydrochloride monohydrate. In some cases, the histidine buffer comprises L-histidine at a concentration of 3.65 mM±0.5 mM and L-histidine monohydrochloride monohydrate at a concentration of 6.35 mM±0.5 mM. In some cases, the histidine buffer comprises L-histidine at a concentration of 3.65 mM±0.2 mM and L-histidine monohydrochloride monohydrate at a concentration of 6.35 mM±0.2 mM. In some embodiments, the formulations contain an acetate buffer (e.g., at any of the concentrations discussed above or herein). In some embodiments, the formulations contain a phosphate buffer (e.g., at any of the concentrations discussed above or herein).

During the antibody purification process it may be desired or necessary to exchange one buffer for another to achieve appropriate excipient concentrations, antibody concentration, pH, etc. Buffer exchange can be accomplished, e.g., by ultrafiltration/diafiltration (UF/DF) using, e.g., a semi-permeable tangential flow filtration membrane. Use of such techniques, however, has the potential to cause the Gibbs-Donnan effect (Bolton et al., 2011, Biotechnol. Prog. 27(1):140-152). The buildup of positive charge on the product side of the membrane during protein concentration is counterbalanced electrically by the preferential movement of positive ions to the opposite side of the membrane. The potential consequence of this phenomenon is that the final concentrations of certain components (e.g., histidine) may be lower than the intended target concentrations of these components due to the electrostatic repulsion of positively charged diafiltration buffer excipients to the positively charged antibody protein during the UF/DF step. Thus, the present invention includes formulations in which the concentration of, e.g., histidine vary from the recited amounts or ranges herein due to the Gibbs-Donnan effect.

Volume exclusion describes the behavior of highly concentrated samples in which a significant portion of the total volume of the solution is taken up by the solute, especially large molecules such as proteins, excluding the solvent from this space. This then decreases the total volume of solvent available for other solutes to be dissolved in, which may result in unequal partition across the ultrafiltration membrane. Thus, the present invention includes formulations in which the concentration of, e.g., histidine may vary from the recited amounts or ranges herein due to the volume exclusion effect.

During the manufacture of the formulations of the present invention, variations in the composition of the formulation may occur. These variations may include the concentration of the active ingredient, the concentration of the excipients, and/or the pH of the formulation. The present invention includes formulations comprising anti-CD20×anti-CD3 bispecific antibodies which are stable and retain potency with up to at least 10% variation in the excipient concentration. For example, included herein are anti-CD20×anti-CD3 bispecific antibody formulations, wherein stability and potency of the formulations is unaffected by ±10%, or ±20% variation in the concentration of antibody, sucrose, histidine buffer and/or polysorbate.

### Stability of the Pharmaceutical Formulations

The pharmaceutical formulations of the present invention exhibit high levels of stability. The term “stable,” as used herein in reference to the pharmaceutical formulations, means that the antibodies within the pharmaceutical formulations retain an acceptable degree of structure and/or function and/or biological activity after storage for a defined amount of time. A formulation may be stable even though the antibody contained therein does not maintain 100% of its structure and/or function and/or biological activity after storage for a defined amount of time. Under certain circumstances, maintenance of about 90%, about 95%, about 96%, about 97%, about 98% or about 99% of an antibody's structure and/or function and/or biological activity after storage for a defined amount of time may be regarded as “stable.”

Stability can be measured by, inter alia, determining the percentage of native antibody remaining in the formulation after storage for a defined amount of time at a given temperature. The percentage of native antibody can be determined by, inter alia, size exclusion chromatography (e.g., size exclusion high performance liquid chromatography [SE-HPLC]). An “acceptable degree of stability,” as that phrase is used herein, means that at least 90% of the native form of the antibody can be detected in the formulation after storage for a defined amount of time at a given temperature. In certain embodiments, at least about 90%, 91%, 92%, 93%, 94%, 95%, 96%, 97%, 98%, 99% or 100% of the native form of the antibody can be detected in the formulation after storage for a defined amount of time at a given temperature. The defined amount of time after which stability is measured can be at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C., or about 45° C. For example, a pharmaceutical formulation may be deemed stable if after 3 months of storage at 5° C., greater than about 90%, 95%, 96% or 97% of native antibody is detected by SE-HPLC. A pharmaceutical formulation may also be deemed stable if after 6 months of storage at 5° C., greater than about 90%, 95%, 96% or 97% of native antibody is detected by SE-HPLC. A pharmaceutical formulation may also be deemed stable if after 9 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 12 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 24 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 36 months of storage at 5° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 3 months of storage at 25° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 6 months of storage at 25° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 9 months of storage at 25° C., greater than about 90%, 95%, 96%, 96.5%, 97%, 97.5%, 98%, 98.5%, 99% or 99.5% of native antibody is detected by SE-HPLC. A pharmaceutical formulation may also be deemed stable if after 1 month of storage at 45° C., greater than about 90%, 91%, 92%, 93%, 94%, 95%, 96%, 97% or 98% of native antibody is detected by SE-HPLC.

Other methods may be used to assess the stability of the formulations of the present invention such as, e.g., differential scanning calorimetry (DSC) to determine thermal stability, controlled agitation to determine mechanical stability, and absorbance at about 350 nm or about 405 nm to determine solution turbidities. For example, a formulation of the present invention may be considered stable if, after 6 or more months of storage at about 5° C. to about 25° C., the change in OD₄₀₅ of the formulation is less than about 0.05 (e.g., 0.04, 0.03, 0.02, 0.01, or less) from the OD₄₀₅ of the formulation at t=0.

Measuring the binding affinity of the antibody to its target may also be used to assess stability. For example, a formulation of the present invention may be regarded as stable if, after storage at e.g., −80° C., −30° C., −20° C., 5° C., 25° C., 37° C., 45° C., etc. for a defined amount of time (e.g., 14 days to 9 months), the anti-CD20×anti-CD3 bispecific antibody contained within the formulation binds to human CD20 and human CD3 with an affinity that is at least 80%, 85%, 90%, 95%, or more of the binding affinity of the antibody prior to said storage. Binding affinity may be determined by any method, such as e.g., ELISA or plasmon resonance. Biological activity may be determined by a CD20 or CD3 activity assay, such as by contacting a cell that expresses CD20 or CD3 with the formulation comprising the anti-CD20×anti-CD3 bispecific antibody. The binding of the antibody to such a cell may be measured directly, such as via FACS analysis.

Stability can be measured, inter alia, by determining the percentage of antibody that forms an aggregate (high molecular weight (HMW) species) within the formulation after storage for a defined amount of time at a defined temperature, wherein stability is inversely proportional to the percent aggregate that is formed. The percentage of aggregated antibody can be determined by, inter alia, size exclusion chromatography (e.g., size exclusion high performance liquid chromatography [SE-HPLC] or size exclusion ultra-performance liquid chromatography [SE-UPLC]). An “acceptable degree of stability”, as that phrase is used herein, means that at most 6% of the antibody is in an aggregated form detected in the formulation after storage for a defined amount of time at a given temperature. In certain embodiments an acceptable degree of stability means that at most about 6%, 5%, 4%, 3%, 2%, 1%, 0.5%, or 0.1% of the antibody can be detected in an aggregate in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., about 35° C., about 37° C. or about 45° C. For example, a pharmaceutical formulation may be deemed stable if after nine months of storage at 5° C., less than about 2%, 1.75%, 1.5%, 1.25%, 1%, 0.75%, 0.5%, 0.25%, or 0.1% of the antibody is detected in an aggregated form. A pharmaceutical formulation may also be deemed stable if after three months of storage at 25° C. and 60% relative humidity, less than about 2%, 1.75%, 1.5%, 1.25%, 1%, 0.75%, 0.5%, 0.25%, or 0.1% of the antibody is detected in an aggregated form. A pharmaceutical formulation may also be deemed stable if after 1 month of storage at 45° C., less than about 6%, 5.9%, 5.8%, 5.7%, 5.6%, 5.5%, 5%, 4.5%, 4%, 3.5%, 3%, 2.5%, 2%, 1.5%, 1%, 0.5%, or 0.1% of the antibody is detected in an aggregated form. A pharmaceutical formulation may also be deemed stable if after three months of storage at −20° C., −30° C., or −80° C. less than about 2%, 1.9%, 1.8%, 1.7%, 1.6%, 1.5%, 1%, 0.5%, or 0.1% of the antibody is detected in an aggregated form.

Stability can be measured, inter alia, by determining the percentage of antibody that migrates in a more acidic fraction during ion exchange (“acidic form”) than in the main fraction of antibody (“main charge form”), wherein stability is inversely proportional to the fraction of antibody in the acidic form. While not wishing to be bound by theory, deamidation of the antibody may cause the antibody to become more negatively charged and thus more acidic relative to the non-deamidated antibody (see, e.g., Robinson, N., Protein Deamidation, PNAS, Apr. 16, 2002, 99(8):5283-5288). The percentage of “acidified” antibody can be determined by ion exchange chromatography (e.g., cation exchange high performance liquid chromatography [CEX-HPLC] or cation exchange ultra-performance liquid chromatography [CEX-UPLC]). An “acceptable degree of stability”, as that phrase is used herein, means that at most 52% of the antibody is in a more acidic form detected in the formulation after storage for a defined amount of time at a defined temperature. In certain embodiments an acceptable degree of stability means that at most about 52%, 50%, 45%, 40%, 35%, 30%, 29%, 28%, 27%, 26%, 25%, 20%, 15%, 10%, 5%, 4%, 3%, 2%, 1%, 0.5%, or 0.1% of the antibody can be detected in an acidic form in the formulation after storage for a defined amount of time at a given temperature. The defined amount of time after which stability is measured can be at least 2 weeks, at least 28 days, at least 1 month, at least 2 months, at least 3 months, at least 4 months, at least 5 months, at least 6 months, at least 7 months, at least 8 months, at least 9 months, at least 10 months, at least 11 months, at least 12 months, at least 18 months, at least 24 months, at least 30 months, at least 36 months, or more. The temperature at which the pharmaceutical formulation may be stored when assessing stability can be any temperature from about −80° C. to about 45° C., e.g., storage at about −80° C., about −30° C., about −20° C., about 0° C., about 4°−8° C., about 5° C., about 25° C., or about 45° C. For example, a pharmaceutical formulation may be deemed stable if after three months of storage at −80° C., −30° C., or −20° C. less than about 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after nine months of storage at 5° C., less than about 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 25° C., less than about 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 37° C., less than about 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody is in a more acidic form. A pharmaceutical formulation may also be deemed stable if after 28 days of storage at 45° C., less than about 52%, 51%, 50%, 49%, 48%, 47%, 46%, 45%, 44%, 43%, 42%, 41%, 40%, 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 10%, 9%, 8%, 7%, 6%, 5%, 4%, 3%, 2%, 1%, 0.5% or 0.1% of the antibody can be detected in a more acidic form.

References to stability of the pharmaceutical formulations “after” a specified period of time are intended to mean that a measurement of a stability parameter (e.g., % native form, % HMW species, or % acidic form) is taken at or about the end of the specific time period, and is not intended to mean that the pharmaceutical formulation necessarily maintains the same degree of stability for the measured parameter thereafter. For example, reference to a particular stability after 9 months means that the measurement of stability was taken at or about 9 months after the start of the study. Additional methods for assessing the stability of an antibody in formulation are demonstrated in the Examples presented below.

As illustrated in the Examples below, the present invention is based, in part, on the discovery that the combination of claimed excipients with a bispecific anti-CD20×anti-CD3 antibody produces a formulation that is stable.

### Exemplary Formulations

According to one aspect of the present invention, the pharmaceutical formulation comprises: (i) a human anti-CD20×anti-CD3 bispecific antibody that specifically binds to human CD20 and human CD3; (ii) a buffer comprising histidine; (iii) an organic co-solvent comprising polysorbate; and (iv) a stabilizer comprising a sugar.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml; (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from about 2 mg/ml to about 200 mg/ml; (ii) histidine at a concentration of from about 5 mM to about 15 mM; (iii) polysorbate 80 at a concentration of from about 0.05% w/v to about 0.15% w/v; and (iv) sucrose at a concentration of from about 5% w/v to about 15% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml; (ii) histidine at a concentration of from about 8 mM to about 12 mM; (iii) polysorbate 80 at a concentration of from about 0.075% w/v to about 0.125% w/v; and (iv) sucrose at a concentration of from about 8% w/v to about 12% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 8 mM to about 12 mM; (iii) polysorbate 80 at a concentration of from about 0.075% w/v to about 0.125% w/v; and (iv) sucrose at a concentration of from about 8% w/v to about 12% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of from about 8 mM to about 12 mM; (iii) polysorbate 80 at a concentration of from about 0.075% w/v to about 0.125% w/v; and (iv) sucrose at a concentration of from about 8% w/v to about 12% w/v, wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from about 2 mg/ml to about 200 mg/ml; (ii) histidine at a concentration of from about 8 mM to about 12 mM; (iii) polysorbate 80 at a concentration of from about 0.075% w/v to about 0.125% w/v; and (iv) sucrose at a concentration of from about 8% w/v to about 12% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml; (ii) histidine at a concentration of 10 mM; (iii) polysorbate 80 at a concentration of 0.1% w/v; and (iv) sucrose at a concentration of 10% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of 10 mM; (iii) polysorbate 80 at a concentration of 0.1% w/v; and (iv) sucrose at a concentration of 10% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from about 2 mg/ml to about 200 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of 10 mM; (iii) polysorbate 80 at a concentration of 0.1% w/v; and (iv) sucrose at a concentration of 10% w/v, wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from about 2 mg/ml to about 200 mg/ml; (ii) histidine at a concentration of 10 mM; (iii) polysorbate 80 at a concentration of 0.1% w/v; and (iv) sucrose at a concentration of 10% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from 2 mg/ml±0.2 mg/ml to 200 mg/ml±20 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 2 mg/ml±0.2 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 20 mg/ml±2 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 100 mg/ml±10 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from 2 mg/ml±0.2 mg/ml to 200 mg/ml±20 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 2 mg/ml±0.2 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 20 mg/ml±2 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 100 mg/ml±10 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of from 2 mg/ml±0.2 mg/ml to 200 mg/ml±20 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 2 mg/ml±0.2 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 20 mg/ml±2 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 100 mg/ml±10 mg/ml, wherein the antibody has heavy chain constant regions of isotype IgG4, and wherein one of the two heavy chains has a modification (e.g. H435R and Y436F by EU numbering) in the CH3 domain that reduces binding to Protein A relative to an unmodified IgG4 CH3 domain; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of from 2 mg/ml±0.2 mg/ml to 100 mg/ml±10 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of 2 mg/ml±0.2 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of 20 mg/ml±2 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of 100 mg/ml±10 mg/ml; (ii) histidine at a concentration of 10 mM±1 mM; (iii) polysorbate 80 at a concentration of 0.1%±0.01 w/v; and (iv) sucrose at a concentration of 10%±1% w/v, wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 2 mg/ml±0.2 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 2 mg/ml±0.2 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 2 mg/ml±0.2 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of 2 mg/ml±0.2 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 20 mg/ml±2 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 20 mg/ml±2 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 20 mg/ml±2 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of 20 mg/ml±2 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 100 mg/ml±10 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 100 mg/ml±10 mg/ml, wherein the antibody has heavy chain constant regions of isotype IgG1 (optionally in which one of the two heavy chains has a modification that reduces Protein A binding relative to an unmodified heavy chain of the same isotype, and optionally in which one or both of the two heavy chains has a chimeric hinge); (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first antigen-binding domain that specifically binds human CD20 comprising a HCVR comprising the amino acid sequence of SEQ ID NO: 4 and a LCVR comprising the amino acid sequence of SEQ ID NO: 6, and a second antigen-binding domain that specifically binds human CD3 comprising a HCVR comprising the amino acid sequence of SEQ ID NO: 5 and a LCVR comprising the amino acid sequence of SEQ ID NO: 6 at a concentration of 100 mg/ml±10 mg/ml, wherein the antibody has heavy chain constant regions of isotype IgG4 (optionally in which one of the two heavy chains has a modification that reduces Protein A binding relative to an unmodified heavy chain of the same isotype, and optionally in which one or both of the two heavy chains has a chimeric hinge); (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In some cases, the stable liquid pharmaceutical formulation comprises (i) a human bispecific antibody that specifically binds to human CD20 and human CD3 and comprises a first heavy chain comprising the amino acid sequence of SEQ ID NO: 1, a second heavy chain comprising the amino acid sequence of SEQ ID NO: 2, and a common light chain comprising the amino acid sequence of SEQ ID NO: 3 at a concentration of 100 mg/ml±10 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

In any of these exemplary formulations, “stable” may be defined as: (a) at least 90% of the antibody has native conformation after 1 month of storage at 45° C., as determined by size exclusion-ultra performance liquid chromatography (SE-UPLC); (b) at least 93% of the antibody has native conformation after 1 month of storage at 45° C., as determined by SE-UPLC; (c) at least 95% of the antibody has native conformation after 1 month of storage at 45° C., as determined by SE-UPLC; (d) at least 95% of the antibody has native conformation after 3 months of storage at 25° C. and 60% relative humidity, as determined by SE-UPLC; (e) at least 97% of the antibody has native conformation after 3 months of storage at 25° C. and 60% relative humidity, as determined by SE-UPLC; (f) at least 95% of the antibody has native conformation after 3 months of storage at 5° C., as determined by SE-UPLC; (g) at least 98% of the antibody has native conformation after 3 months of storage at 5° C., as determined by SE-UPLC; (h) the formulation contains no more than 6% high molecular weight (HMW) species after 1 month of storage at 45° C., as determined by SE-UPLC; (i) the formulation contains no more than 5.6% HMW species after 1 month of storage at 45° C., as determined by SE-UPLC; (j) the formulation contains no more than 2% HMW species after 3 months of storage at 25° C. and 60% relative humidity, as determined by SE-UPLC; (k) the formulation contains no more than 1% HMW species after 3 months of storage at 25° C. and 60% relative humidity, as determined by SE-UPLC; (l) the formulation contains no more than 1.5% HMW species after 3 months of storage at 5° C., as determined by SE-UPLC; and/or (m) the formulation contains no more than 1% HMW species after 9 months of storage at 5° C., as determined by SE-UPLC.

In any of these exemplary formulations, the bispecific antibody may include a modification in one or both heavy chains to facilitate purification of the bispecific antibody (i.e., the heterodimer) from homodimeric impurities. In some embodiments, the bispecific antibodies include first and second heavy chains (i.e., the heavy chain of the anti-CD20 binding arm, and the heavy chain of the anti-CD3 binding arm) that are identical (e.g., both of isotype IgG1 or IgG4) except for a modification in the CH3 domain of one or the other heavy chain that reduces binding of the bispecific antibody to Protein A as compared to an antibody lacking the modification. In some cases, the CH3 domain of the first heavy chain (e.g., of the anti-CD20 binding arm) binds Protein A and the CH3 domain of the second heavy chain (e.g., of the anti-CD3 binding arm) contains a mutation that reduces or abolishes Protein A binding. In some cases, the mutation is a H435R modification (by EU numbering; H95R by IMGT exon numbering). In some cases, the mutation is a H435R modification (by EU numbering; H95R by IMGT exon numbering) and a Y436F modification (by EU numbering; Y96F by IMGT). Further modifications that may be found within the second CH3 domain include: D356E, L358M, N384S, K392N, V397M, and V422I by EU (D16E, L18M, N44S, K52N, V57M, and V82I by IMGT) in the case of IgG1 CH3 domains; and Q355R, N384S, K392N, V397M, R409K, E419Q, and V422I by EU (Q15R, N44S, K52N, V57M, R69K, E79Q, and V82I by IMGT) in the case of IgG4 CH3 domains.

Additional non-limiting examples of pharmaceutical formulations encompassed by the present invention are set forth elsewhere herein, including the working Examples presented below.

### Containers and Methods of Administration

The pharmaceutical formulations of the present invention may be contained within any container suitable for storage of medicines and other therapeutic compositions. For example, the pharmaceutical formulations may be contained within a sealed and sterilized plastic or glass container having a defined volume such as a vial, ampule, syringe, cartridge, bottle or IV bag. Different types of vials can be used to contain the formulations of the present invention including, e.g., clear and opaque (e.g., amber) glass or plastic vials. Likewise, any type of syringe can be used to contain and/or administer the pharmaceutical formulations of the present invention. In some embodiments, the pharmaceutical formulation is contained in a prefilled syringe. In some embodiments, the pharmaceutical formulation is contained in a prefilled staked needle syringe.

The pharmaceutical formulations of the present invention may be contained within “normal tungsten” syringes or “low tungsten” syringes. As will be appreciated by persons of ordinary skill in the art, the process of making glass syringes generally involves the use of a hot tungsten rod which functions to pierce the glass thereby creating a hole from which liquids can be drawn and expelled from the syringe. This process results in the deposition of trace amounts of tungsten on the interior surface of the syringe. Subsequent washing and other processing steps can be used to reduce the amount of tungsten in the syringe. As used herein, the term “normal tungsten” means that the syringe contains greater than 500 parts per billion (ppb) of tungsten. The term “low tungsten” means that the syringe contains less than 500 ppb of tungsten. For example, a low tungsten syringe, according to the present invention, can contain less than about 490, 480, 470, 460, 450, 440, 430, 420, 410, 390, 350, 300, 250, 200, 150, 100, 90, 80, 70, 60, 50, 40, 30, 20, 10 or fewer ppb of tungsten.

The rubber plungers used in syringes, and the rubber stoppers used to close the openings of vials, may be coated to prevent contamination of the medicinal contents of the syringe or vial and/or to preserve their stability. Thus, pharmaceutical formulations of the present invention, according to certain embodiments, may be contained within a syringe that comprises a coated plunger, or within a vial that is sealed with a coated rubber stopper. For example, the plunger or stopper may be coated with a fluorocarbon film. Examples of coated stoppers and/or plungers suitable for use with vials and syringes containing the pharmaceutical formulations of the present invention are mentioned in, e.g., U.S. Pat. Nos. 4,997,423; 5,908,686; 6,286,699; 6,645,635; and 7,226,554, the contents of which are incorporated by reference herein in their entireties. Particular exemplary coated rubber stoppers and plungers that can be used in the context of the present invention are commercially available under the tradename “FluroTec®,” available from West Pharmaceutical Services, Inc. (Lionville, Pa.). FluroTec® is an example of a flurocarbon coating used to minimize or prevent drug product from adhering to the rubber surfaces. According to certain embodiments of the present invention, the pharmaceutical formulations may be contained within a low tungsten syringe that comprises a fluorocarbon-coated plunger.

The pharmaceutical formulations can be administered to a patient by parenteral routes such as injection (e.g., subcutaneous, intravenous, intramuscular, intraperitoneal, etc.) or percutaneous, mucosal, nasal, pulmonary and/or oral administration. Numerous reusable pen and/or autoinjector delivery devices can be used to subcutaneously deliver the pharmaceutical formulations of the present invention. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PROT″, OPTIPEN STARLET™, and OPTICLIK™ (sanofi-aventis, Frankfurt, Germany), to name only a few. Examples of disposable pen and/or autoinjector delivery devices having applications in subcutaneous delivery of a pharmaceutical composition of the present invention include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L.P.), and the HUMIRA™ Pen (Abbott Labs, Abbott Park, Ill.), to name only a few. In some cases, the pharmaceutical formulation is contained in a syringe specifically adapted for use with an autoinjector.

The use of a microinfusor to deliver the pharmaceutical formulations of the present invention is also contemplated herein. As used herein, the term “microinfusor” means a subcutaneous delivery device designed to slowly administer large volumes (e.g., up to about 2.5 mL, about 3.0 mL or more) of a therapeutic formulation over a prolonged period of time (e.g., about 10, 15, 20, 25, 30 or more minutes). See, e.g., U.S. Pat. Nos. 6,629,949; 6,659,982; and Meehan et al., J. Controlled Release 46:107-116 (1996). Microinfusors are particularly useful for the delivery of large doses of therapeutic proteins contained within high concentration (e.g., about 100, 125, 150, 175, 200 or more mg/mL) and/or viscous solutions.

In certain embodiments, the pharmaceutical formulation is administered via an IV drip, such that the formulation is diluted in an IV bag containing a physiologically acceptable solution. In one embodiment, the pharmaceutical composition is a compounded sterile preparation in an intravenous infusion bag, such that a single dose of drug product is diluted into 100 mL, 250 mL (or other like amount suitable for intravenous drip delivery) of a physiological buffer (e.g., 0.9% saline).

The pharmaceutical formulations of the present invention can also be contained in a unit dosage form. The term “unit dosage form,” as used herein, refers to a physically discrete unit suitable as a unitary dosage for the patient to be treated, each unit containing a predetermined quantity of active compound calculated to produce the desired therapeutic effect in association with the required pharmaceutical carrier, diluent, or excipient. In various embodiments, the unit dosage form is contained within a container as discussed herein. Actual dosage levels of the active ingredient (e.g., an anti-CD20×anti-CD3 bispecific antibody) in the formulations of the present invention may be varied so as to obtain an amount of the active ingredient which is effective to achieve the desired therapeutic response for a particular patient, composition, and mode of administration, without adverse effect to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts. The term “diluent” as used herein refers to a solution suitable for altering or achieving an exemplary or appropriate concentration or concentrations as described herein.

In various embodiments, the unit dosage form contains an amount of the active ingredient (e.g., an anti-CD20×anti-CD3 bispecific antibody) intended for a single use. In various embodiments, the amount of the active ingredient in the unit dosage form is from about 0.1 mg to about 5000 mg, from about 100 mg to about 1000 mg, and from about 100 mg to about 500 mg, from about 100 mg to about 400 mg, from about 100 mg to about 200 mg, from about 250 mg to about 350 mg, from about 125 mg to about 175 mg, from about 275 mg to about 325 mg, from about 1 mg to about 250 mg, from about 1 mg to about 100 mg, from about 1 mg to about 50 mg, from about 1 mg to about 25 mg, from about 1 mg to about 10 mg, from about 1 mg to about 5 mg, or ranges or intervals thereof. Ranges intermediate to the above recited amounts, for example, from about 2 mg to about 100 mg or 2 mg to 20 mg, are also intended to be part of this invention. For example, ranges of values using a combination of any of the above recited values (or values contained within the above recited ranges) as upper and/or lower limits are intended to be included. In a particular embodiment, the formulation often is supplied as a liquid in unit dosage form. In some embodiments, the unit dosage form contains from 2 to 2.5 mg, or from 10 to 11 mg, from 20 to 25 mg, from 80 to 90 mg, 100 to 125 mg, 160 to 180 mg, 200 to 225, or 320 to 360 mg. In some embodiments, a unit dosage form according to the present invention is suitable for subcutaneous administration to a patient (e.g., a unit dosage form containing the antibody at a concentration of about 100 mg/ml or about 160 mg/ml).

In one embodiment, the present invention provides a unit dosage form comprising about 2 mg of the antibody in a stable formulation wherein the formulation comprises the antibody at a concentration of 2 mg/ml±0.2 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3. In one embodiment, the present invention provides a unit dosage form comprising about 20 mg, about 80 mg, about 160 mg or about 320 mg of the antibody in a stable formulation wherein the formulation comprises the antibody at a concentration of 20 mg/ml±2 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3. In one embodiment, the present invention provides a unit dosage form comprising about 100 mg, about 200 mg, about 300 mg or about 400 mg of the antibody in a stable formulation wherein the formulation comprises the antibody at a concentration of 100 mg/ml±10 mg/ml; (ii) L-histidine at a concentration of 0.57 mg/mL±0.1 mg/mL; (iii) L-histidine monohydrochloride monohydrate at a concentration of 1.33 mg/mL±0.1 mg/mL; (iv) polysorbate 80 at a concentration of 1 mg/mL±0.1 mg/mL; and (v) sucrose at a concentration of 100 mg/mL±10 mg/mL, in water wherein the formulation has a pH of 5.8±0.3.

The present invention also includes methods of preparing a unit dosage form. In an exemplary embodiment, a method for preparing a pharmaceutical unit dosage form includes combining the formulation of any of foregoing embodiments in a suitable container (e.g., those containers discussed herein).

In various embodiments, the pharmaceutical formulations are contained in containers (e.g., a vial or a prefilled syringe) that may contain a headspace gas with less than 5% of an oxidizing gas (e.g., oxygen) by volume. The concentration of oxidizing gas (e.g., oxygen) in the headspace of the container may be less than 4.5%, less than 4%, less than 3.5%, less than 3%, less than 2.5%, less than 2%, or less than 1.5% in various embodiments. In one embodiment, the concentration of the oxidizing gas (e.g., oxygen) in the headspace is less than about 1%. In one embodiment, the concentration of the oxidizing gas (e.g. oxygen) in the headspace is no more than about 0.5%. In one embodiment, the concentration of the oxidizing gas (e.g. oxygen) in the headspace is no more than about 0.1%. In various embodiments, the concentration of the oxidizing gas (e.g., oxygen) in the headspace of the drug product container is less than 0.9%, less than 0.8%, less than 0.7%, less than 0.6%, less than 0.5%, less than 0.4%, less than 0.3%, less than 0.2%, or less than 0.1%. In some cases, the concentration of oxygen in the headspace gas is from about 0.01% to about 1.5%. In some cases, the concentration of oxygen in the headspace gas is from about 0.75% to about 1.25%. In some cases, the concentration of oxygen in the headspace gas is from about 0.05% to about 0.15%. In various embodiments, the oxidizing gas (e.g., oxygen) in the headspace is replaced, or substantially replaced, with an inert gas, such as nitrogen, argon, helium, xenon, neon, krypton or radon. In one embodiment, the non-oxidizing gas is nitrogen. In one embodiment, the non-oxidizing gas is argon.

### Therapeutic Uses of the Pharmaceutical Formulations

The pharmaceutical formulations of the present invention are useful, inter alia, for the treatment, prevention and/or amelioration of any disease or disorder associated with a cell expressing human CD20. Exemplary, non-limiting diseases and disorders that can be treated by the administration of the pharmaceutical formulations of the present invention include B-cell cancers, such as non-Hodgkin's lymphomas, e.g., follicular lymphoma, diffuse large B-cell lymphoma, mantle cell lymphoma and marginal zone lymphoma.

The therapeutic methods of the present invention comprise administering to a subject any formulation comprising an anti-CD20×anti-CD3 bispecific antibody as disclosed herein. The subject to which the pharmaceutical formulation is administered can be, e.g., any human or non-human animal that is in need of such treatment. For example, the subject can be an individual that is diagnosed with, or who is deemed to be at risk of being afflicted by any of the aforementioned diseases or disorders. The present invention further includes the use of any of the pharmaceutical formulations disclosed herein in the manufacture of a medicament for the treatment of any disease or disorder associated with a cell expressing human CD20, including any of the above mentioned exemplary diseases, disorders and conditions.

In some embodiments, the present invention provides kits comprising a pharmaceutical formulation (e.g., a container with the formulation or a unit dosage form), as discussed herein, and packaging or labeling (e.g., a package insert) with instructions to use the pharmaceutical formulation for the treatment of a disease or disorder, as discussed above. In some cases, the instructions provide for use of a unit dosage form, as discussed herein, for the treatment of a disease or disorder.

A summary of the sequences and the corresponding SEQ ID NOs referenced herein is shown in Table 1, below.

TABLE 1
Summary of Sequences

| SEQ ID NO: | Description |
|---|---|
| 1 | Anti-CD20 Heavy Chain |
| 2 | Anti-CD3 Heavy Chain |
| 3 | Common Anti-CD20 and Anti-CD3 Light Chain |
| 4 | Anti-CD20 HCVR |
| 5 | Anti-CD3 HCVR |
| 6 | Common Anti-CD20 and Anti-CD3 LCVR |
| 7 | Anti-CD20 HCDR1 |
| 8 | Anti-CD20 HCDR2 |
| 9 | Anti-CD20 HCDR3 |
| 10 | Anti-CD3 HCDR1 |
| 11 | Anti-CD3 HCDR2 |
| 12 | Anti-CD3 HCDR3 |
| 13 | Common Anti-CD20 and Anti-CD3 LCDR1 |
| 14 | Common Anti-CD20 and Anti-CD3 LCDR2 |
| 15 | Common Anti-CD20 and Anti-CD3 LCDR3 |
| 16 | IgG4 Heavy Chain Constant Region |
| 17 | IgG4 Heavy Chain Constant Region with H435R/Y436F |
| 18 | IgG1 Heavy Chain Constant Region |
| 19 | IgG1 Heavy Chain Constant Region with H435R/Y436F |

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Development of Stable Liquid Anti-CD20×Anti-CD3 Bispecific Antibody Formulations

Formulation development activities involved assessment of buffers, pH, organic co-solvents, surfactants, and thermal stabilizers to identify excipients that enhance protein stability. Results generated from these studies, as well as those discussed in Example 2, were used to develop a stable liquid formulation suitable for clinical use.

The effect of buffer and pH on the thermal stability of REGN1979 was examined in liquid formulations by incubating 15 mg/mL REGN1979 at 45° C. for 28 days in a series of buffer systems at varying pH ranges. The following pH and buffer systems were studied: acetate (pH 4.5-5.5), histidine (pH 5.5-6.5), and phosphate (pH 6.0-7.0). Based on results from SE-UPLC analysis, maximum protein stability was observed when REGN1979 was formulated between pH 5.5 and pH 6.0 in histidine buffer (Table 2). Based on results from CEX-UPLC analysis, maximum protein stability was observed when REGN1979 was formulated between pH 5.5 and pH 6.0 in histidine buffer or between pH 4.5 and 5.0 in acetate buffer. These analyses also revealed that fragmentation (i.e., formation of low molecular weight species), formation of HMW species, and charge variants were the main degradation pathways. Histidine buffer was selected as the formulation buffer for the drug product (DP) formulations because it minimized the formation of high molecular weight species, the degradation pathway of greater concern. A pH of 5.8 was chosen for the DP formulations because formation of high molecular weight species and charge variants were minimized at this pH. Based on these results, as well as those discussed in Example 2, 10 mM histidine buffer, at pH 5.8, was chosen for the REGN1979 DP formulations.

Thermal stabilizers such as sucrose are often added to antibody formulations to increase the thermal stability of the protein. 25 mg/mL REGN1979 in a liquid formulation exhibited improved stability when formulated with 10% sucrose and incubated under accelerated conditions (Table 3). After incubation at 45° C. for 28 days, HMW species increased by 0.6% in the formulation containing 10% sucrose compared to a 1.4% increase in the formulation without sucrose. Based on these results, as well as those discussed in Example 2, sucrose was chosen as the thermal stabilizer for the REGN1979 DP formulation.

Organic co-solvents, such as surfactants, are often added to antibody formulations to protect the protein from agitation-induced aggregation. The effect of surfactants on the agitation stress stability and thermal stability of 25 mg/mL REGN1979 was examined. The following surfactants were evaluated: 0.1% polysorbate 20 and 0.1% polysorbate 80 in this example. The results of agitation stress stability and thermal stability studies are summarized in Table 4 and Table 5, respectively. REGN1979 was unstable when agitated by vortexing for 120 minutes in the absence of a surfactant. In the absence of surfactant, the formulation exhibited a 1.5% increase in HMW species as determined by SE-UPLC (Table 4). Both of the surfactants that were tested protected REGN1979 from agitation-induced instability to a similar extent (Table 4). Moreover, both of the surfactants that were tested decreased the thermal stability of REGN1979 to a similar extent, which manifested as an increase in HMW species, LMW species, and basic charged species (Table 5). Based on these results, as well as those discussed in Example 2, polysorbate 80 was chosen as the surfactant for the REGN1979 DP formulations.

REGN1979 exhibited maximal stability when formulated in the presence of histidine, polysorbate 80, and sucrose at pH 5.8. The main degradation pathways identified during the development of REGN1979 liquid formulations were the formation of high and low molecular weight species and charge variants. Based on the results of these experiments, and those discussed in Example 2, an aqueous buffered formulation containing 10 mM histidine, pH 5.8, 0.1% (w/v) polysorbate 80, 10% (w/v) sucrose, and from 2-160 mg/mL (e.g., 2 mg/ml, 20 mg/ml or 100 mg/ml) REGN1979 was determined to be most stable.

TABLE 2
Effect of Buffer and pH on the Stability of 15 mg/mL REGN1979 Incubated at 45° C. for 28 Days
Formulation 15 mg/mL REGN1979, 10 mM Buffer
Fill Volume 0.4 mL
Container/Closure 2 mL Type 1 borosilicate glass vial with a FluroTec ® coated 4432/50 elastomeric stopper

| | | | Turbidity | % Protein | Change in Purity | | | Change in Charged Variants | | |
|---|---|---|---|---|---|---|---|---|---|---|
| | | | (Increase | Recovered | by SE-UPLC^{a} | | | by CEX-UPLC^{a} | | |
| | FDG Lot | Color and | in OD at | by | % | % | % | % | % | % |
| pH/Buffer | Number | Appearance | 405 nm) | RP-UPLC | HMW | Native | LMW | Acidic | Main | Basic |
| pH 4.5, Acetate | L13-529 | Pass | 0.01 | 98 | 5.2 | −6.0 | 0.8 | −0.8 | −7.4 | 8.2 |
| pH 5.0, Acetate | L13-528 | Pass | 0.00 | 99 | 1.5 | −1.9 | 0.4 | 6.3 | −6.0 | −0.3 |
| pH 5.5, Acetate | L13-527 | Pass | 0.00 | 97 | 1.5 | −1.8 | 0.3 | 11.8 | −14.1 | 2.3 |
| pH 5.5, Histidine | L13-546 | Pass | 0.00 | 100 | 1.5 | −1.8 | 0.4 | 7.9 | −7.1 | −0.8 |
| pH 6.0, Histidine | L13-545 | Pass | 0.00 | 98 | 1.2 | −1.6 | 0.3 | 13.5 | −9.1 | −4.5 |
| pH 6.5, Histidine | L13-544 | Pass | 0.01 | 101 | 2.0 | −2.6 | 0.6 | 21.7 | −16.6 | −5.1 |
| pH 6.0, | L13-523 | Pass | 0.01 | 100 | 1.5 | −1.8 | 0.3 | 17.1 | −11.6 | −5.5 |
| Phosphate | | | | | | | | | | |
| pH 6.5, | L13-522 | Pass | 0.01 | 97 | 2.4 | −2.8 | 0.4 | 26.0 | −21.0 | −5.1 |
| Phosphate | | | | | | | | | | |
| pH 7.0, | L13-521 | Pass | 0.21 | 96 | 2.8 | −3.2 | 0.4 | 41.1 | −33.2 | −7.9 |
| Phosphate | | | | | | | | | | |

^{a}Reported as a change in purity relative to the starting material. The starting material (no incubation) contains ≥93.3% native peak by SE-UPLC and ≥64.6% main peak by CEX-UPLC in all 9 formulations

TABLE 3
Effect of Sucrose on the Stability of 25 mg/mL REGN1979 Incubated at 45° C. for 28 Days
Formulation 25 mg/mL REGN1979, 10 mM histidine, pH 5.5
Fill Volume 0.4 mL
Container/Closure 2 mL Type 1 borosilicate glass vial with a FluorTec ® coated 4432/50 elastomeric stopper

| | | | Turbidity | | % Protein | Change in Purity | | | Change in Charge | | |
|---|---|---|---|---|---|---|---|---|---|---|---|
| | | | (Increase | | Recovered | by SE-UPLC^{a} | | | Variants by CEX-UPLC^{a} | | |
| | FDG Lot | Color and | in OD at | | by | % | % | % | % | % | % |
| Stabilizer | Number | Appearance | 405 nm) | pH | RP-UPLC | HMW | Native | LMW | Acidic | Main | Basic |
| No sucrose | L13-676 | Pass | 0.00 | 5.4 | 92 | 1.4 | −1.9 | 0.6 | 9.0 | −11.1 | 2.0 |
| 10% (w/v) sucrose | L13-677 | Pass | 0.01 | 5.6 | 95 | 0.6 | −0.9 | 0.0 | 8.7 | −11.7 | 3.1 |

^{a}Reported as a change in purity relative to the starting material. The starting material (no incubation) contains ≥97.1% native peak by SE-UPLC and ≥65.4% main peak by CEX-UPLC in both formulations

TABLE 4
Effect of Surfactants on the Stability of 25 mg/mL REGN1979 Following Agitation (120 Minutes of Vortexing)
Formulation 25 mg/mL REGN1979, 10 mM histidine, pH 5.5, and 10% (w/v) sucrose
Fill Volume 0.4 mL
Container/Closure 2 mL Type 1 borosilicate glass vial with a FluorTec ® coated 4432/50 elastomeric stopper

| | | | Turbidity | | % Protein | Change in Purity | | | Change in Charge Variants | | |
|---|---|---|---|---|---|---|---|---|---|---|---|
| | | | (Increase | | Recovered | by SE-UPLC^{a} | | | by CEX-UPLC^{a} | | |
| | FDG Lot | Color and | in OD at | | by | % | % | % | % | | % |
| Surfactant | Number | Appearance | 405 nm) | pH | RP-UPLC | HMW | Native | LMW | Acidic | % Main | Basic |
| No surfactant | L13-677 | Pass | 0.00 | 5.5 | 99 | 1.5 | −1.4 | −0.1 | −0.6 | −0.2 | 0.8 |
| 0.1% (w/v) | L13-678 | Pass | 0.00 | 5.5 | 100 | −0.2 | 0.3 | −0.1 | 0.2 | −0.1 | −0.1 |
| polysorbate 20 | | | | | | | | | | | |
| 0.1% (w/v) | L13-679 | Pass | 0.00 | 5.5 | 103 | 0.0 | 0.0 | 0.0 | 0.0 | 0.0 | −0.2 |
| polysorbate 80 | | | | | | | | | | | |

^{a}Reported as a change in purity relative to the starting material. The starting material (no incubation) contains ≥97.1% native peak by SE-UPLC and ≥65.4% main peak by CEX-UPLC in all 3 formulations

TABLE 5
Effect of Surfactants on the Stability of 25 mg/mL REGN1979 Incubated at 45° C. for 28 Days
Formulation 25 mg/mL REGN1979, 10 mM histidine, pH 5.5, and 10% (w/v) sucrose
Fill Volume 0.4 mL
Container/Closure 2 mL Type 1 borosilicate glass vial with a FluorTec ® coated 4432/50 elastomeric stopper

| | | | | | | Change in Relative | | | Change in Relative Abundance | | |
|---|---|---|---|---|---|---|---|---|---|---|---|
| | | | Turbidity | | % Protein | Abundance of Molecular | | | of Charge Variant | | |
| | | | (Increase | | Recovered | Weight Species by SE-UPLC^{a} | | | Forms by CEX-UPLC^{a} | | |
| Co-solvent/ | FDG Lot | Color and | in OD at | | by RP- | % | % | % | % | % | % |
| Surfactant | Number | Appearance | 405 nm) | pH | UPLC | HMW | Native | LMW | Acidic | Main | Basic |
| No surfactant | L13-677 | Pass | 0.01 | 5.6 | 95 | 0.6 | −0.9 | 0.0 | 8.7 | −11.7 | 3.1 |
| 0.1% (w/v) | L13-678 | Pass | 0.01 | 5.6 | 95 | 1.9 | −2.5 | 0.6 | 7.7 | −16.1 | 8.4 |
| polysorbate 20 | | | | | | | | | | | |
| 0.1% (w/v) | L13-679 | Pass | 0.01 | 5.6 | 97 | 2.0 | −2.7 | 0.7 | 8.8 | −17.2 | 8.2 |
| polysorbate 80 | | | | | | | | | | | |

^{a}Reported as a change in purity relative to the starting material. The starting material (no incubation) contains ≥97.1% native peak by SE-UPLC and ≥65.4% main peak by CEX-UPLC in all 3 formulations

### Example 2: Storage and Stress Stability of Formulations

Research stability studies were conducted to evaluate the storage, accelerated, and stress stability of REGN1979 drug product (DP) formulations. The DP used in the research stability studies was created by filling 1.2 mL or 5.5 mL of drug substance in 2 mL or 10 mL Type 1 glass vials, respectively, followed by a nitrogen overlay process. The DP was incubated under several high temperature conditions. These accelerated conditions were selected to simulate the conditions which the DP may be subjected to during manufacturing and handling and to elucidate the degradation pathways for REGN1979 DP with nitrogen overlay.

Storage Stability: Currently, 9 months of research stability data is available for the 2 mL vial and 10 mL vial DP. For both DP presentations, the REGN1979 DP was physically and chemically stable when stored at 5° C. for 9 months (Table 6 and Table 7). No appreciable change in the physical or chemical stability was detected in any of the monitored attributes.

Accelerated Stability: Results from the analysis of the REGN1979 2 mL vial and 10 mL vial DP following incubation under accelerated conditions are provided in Table 8 and Table 9, respectively. For both DP presentations, no appreciable degradation was observed when the protein was incubated for one month at 25° C., indicating that the both REGN1979 DPs can be exposed to short periods of time at room temperature. After incubation for 28 days at 45° C., appreciable formation of HMW, LMW, and charge variants were detected. The results from this accelerated condition demonstrated that the formation of HMW, LMW, and charge variants were the main degradation pathway for the 2 mL vial and 10 mL vial DP.

Stress Stability: Stress stability results from the REGN1979 2 mL vial and 10 mL vial DP are provided in Table 10 and Table 11, respectively. Both DP presentations were physically and chemically stable when agitated (vortexed at ambient temperature) for 120 minutes or when subjected to 8 freeze/thaw cycles (freezing at −30° C. and thawing at room temperature). No appreciable change in the physical or chemical stability was detected in any of the monitored attributes.

TABLE 6
Research Stability of REGN1979 Drug Product Stored at 5° C.
Formulation 2 mg/mL REGN1979, 10 mM histidine, 10% (w/v) sucrose, 0.1% (w/v)
polysorbate 80, pH 5.8 (with nitrogen overlay)
Fill Volume 1.2 mL
Container/Closure 2 mL Type 1 borosilicate glass vials with a 13 mm FluroTec ® coated
West V2-F451W 4432/50 GRY B2-TR stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | Pass | Pass | |
| Turbidity (Increase in OD at | | 0.00 | 0.00 | 0.00 | 0.00 | 0.00 | 0.00 | 0.00 | 0.00 | |
| 405 nm) | | | | | | | | | | |
| pH | | 5.8 | 5.8 | 5.8 | 5.9 | 5.8 | 5.8 | 5.8 | 5.9 | |
| Particulate | 2-10 μm | 272 | NR | NR | 818 | NR | 2632 | NR | 3169 | |
| Analysis by | ≥10 μm | 7 | NR | NR | 13 | NR | 27 | NR | 21 | |
| MFI | ≥25 μm | 0 | NR | NR | 0 | NR | 0 | NR | 4 | |
| (particles/mL) | | | | | | | | | | |
| % Protein Recovered by RP- | | 100 | 104 | 101 | 100 | 101 | 101 | 101 | 99 | |
| UPLC | | | | | | | | | | |
| Purity by | Non-reduced; | 97.7 | NR | NR | 96.4 | NR | 96.8 | NR | 96.8 | |
| MCE | % main peak | | | | | | | | | |
| | Reduced; | 99.7 | NR | NR | 99.9 | NR | 99.9 | NR | 99.9 | |
| | % heavy + light | | | | | | | | | |
| | chain | | | | | | | | | |
| Purity by | % HMW | 0.6 | 0.6 | 0.6 | 0.6 | 0.6 | 0.5 | 0.6 | 0.6 | |
| SE-UPLC | % Native | 99.4 | 99.4 | 99.4 | 99.4 | 99.4 | 99.5 | 99.2 | 99.4 | |
| | % LMW | 0.0 | 0.0 | 0.0 | 0.0 | 0.0 | 0.0 | 0.3 | 0.0 | |
| Charge | % Acidic | 14.0 | 14.4 | 16.3 | 14.1 | 13.6 | 15.8 | 14.3 | 16.1 | |
| Variant | % Main | 39.1 | 37.8 | 35.0 | 35.2 | 34.6 | 34.4 | 36.3 | 35.2 | |
| Analysis by | % Basic | 46.9 | 47.8 | 48.6 | 50.7 | 51.8 | 49.8 | 49.4 | 48.8 | |
| CEX-UPLC | | | | | | | | | | |
| Charge | % Acidic | 20.3 | NR | NR | 19.8 | NR | 21.1 | NR | 21.3 | |
| Variant | % Main | 70.8 | NR | NR | 71.7 | NR | 69.6 | NR | 68.9 | |
| Analysis by | % Basic | 8.9 | NR | NR | 8.5 | NR | 9.3 | NR | 9.8 | |
| iCIEF | | | | | | | | | | |

| | | | | | | | | |
|---|---|---|---|---|---|---|---|---|
| % Relative Potency by Bioassay | 92 | NR | NR | 118 | NR | 108 | 108 | 77 |

CEX, cation exchange; DS, drug substance; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 7
Research Stability of REGN1979 Drug Product Stored at 5° C.
Formulation 2 mg/mL REGN1979, 10 mM histidine, 10% (w/v) sucrose,
0.1% (w/v) polysorbate 80, pH 5.8 (with nitrogen overlay)
Fill Volume 5.5 mL
Container/Closure 10 mL Type 1 borosilicate glass vials with a 20 mm FluroTec ®
coated West V10W-F597W 4432/50 GRY B2-TR stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | Pass | Pass | |
| Turbidity (Increase in OD at | | 0.00 | 0.00 | 0.00 | 0.00 | 0.00 | 0.00 | 0.00 | 0.00 | |
| 405 nm) | | | | | | | | | | |
| pH | | 5.8 | 5.8 | 5.8 | 5.9 | 5.9 | 5.8 | 5.8 | 5.9 | |
| Particulate | 2-10 μm | 299 | NR | NR | 403 | NR | 865 | NR | 3810 | |
| Analysis by | ≥10 μm | 9 | NR | NR | 7 | NR | 23 | NR | 6 | |
| MFI | ≥25 μm | 3 | NR | NR | 3 | NR | 2 | NR | 2 | |
| (particles/mL) | | | | | | | | | | |
| % Protein Recovered by RP- | | 100 | 105 | 101 | 100 | 101 | 101 | 101 | 98 | |
| UPLC | | | | | | | | | | |
| Purity by | Non-reduced; | 96.8 | NR | NR | 97.2 | NR | 97.0 | NR | 96.9 | |
| MCE | % main peak | | | | | | | | | |
| | Reduced; | 99.5 | NR | NR | 99.7 | NR | 99.9 | NR | 99.9 | |
| | % heavy + light | | | | | | | | | |
| | chain | | | | | | | | | |
| Purity by | % HMW | 0.6 | 0.6 | 0.6 | 0.6 | 0.6 | 0.5 | 0.6 | 0.6 | |
| SE-UPLC | % Native | 99.4 | 99.4 | 99.4 | 99.4 | 99.4 | 99.5 | 99.2 | 99.4 | |
| | % LMW | 0.0 | 0.0 | 0.0 | 0.0 | 0.0 | 0.0 | 0.3 | 0.0 | |
| Charge | % Acidic | 14.8 | 15.5 | 15.8 | 14.0 | 13.2 | 15.6 | 14.8 | 15.0 | |
| Variant | % Main | 38.5 | 37.7 | 34.6 | 35.2 | 35.5 | 34.8 | 35.2 | 35.9 | |
| Analysis by | % Basic | 46.8 | 46.8 | 49.6 | 50.8 | 51.3 | 49.6 | 49.9 | 49.1 | |
| CEX-UPLC | | | | | | | | | | |
| Charge | % Acidic | 21.8 | NR | NR | 20.5 | NR | 21.0 | NR | 20.9 | |
| Variant | % Main | 70.2 | NR | NR | 70.4 | NR | 69.6 | NR | 69.3 | |
| Analysis by | % Basic | 8.0 | NR | NR | 9.2 | NR | 9.5 | NR | 9.8 | |
| iCIEF | | | | | | | | | | |

| | | | | | | | | |
|---|---|---|---|---|---|---|---|---|
| % Relative Potency by Bioassay | 56 | NR | NR | 95 | NR | 123 | NR | 78 |

CEX, cation exchange; DS, drug substance; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 8
Research Stability of REGN1979 Drug Product-Effect of Accelerated
Conditions
Formulation 2 mg/mL REGN1979, 10 mM histidine, 10% (w/v) sucrose,
0.1% (w/v) polysorbate 80, pH 5.8 (with nitrogen overlay)
Fill Volume 1.2 mL
Container/Closure 2 mL Type 1 borosilicate glass vials with a 13 mm FluroTec ®
coated West V2-F451W 4432/50 GRY B2-TR stoppers

| | | | 25° C./60% RH Storage | | | | | |
|---|---|---|---|---|---|---|---|---|
| | | | (months) | | | 45° C. Storage (months) | | |
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 | | 0.00 | 0.00 | 0.00 | 0.00 | 0.00 | 0.00 | 0.01 |
| nm) | | | | | | | | |
| pH | | 5.8 | 5.8 | 5.8 | 5.9 | 5.8 | 5.8 | 5.8 |
| Particulate | 2-10 μm | 272 | NR | NR | 643 | NR | NR | 996 |
| Analysis by | ≥10 μm | 7 | NR | NR | 15 | NR | NR | 38 |
| MFI | ≥25 μm | 0 | NR | NR | 0 | NR | NR | 5 |
| (particles/mL) | | | | | | | | |
| % Protein Recovered by RP- | | 100 | 104 | 101 | 100 | 99 | 105 | 100 |
| UPLC | | | | | | | | |
| Purity by | Non-reduced; | 97.7 | NR | NR | 95.1 | NR | NR | 93.4 |
| MCE | % main peak | | | | | | | |
| | Reduced; | 99.7 | NR | NR | 99.2 | NR | NR | 97.3 |
| | % heavy + light | | | | | | | |
| | chain | | | | | | | |
| Purity by | % HMW | 0.6 | 0.6 | 0.6 | 0.6 | 0.6 | 0.9 | 2.6 |
| SE-UPLC | % Native | 99.4 | 99.4 | 99.4 | 99.4 | 99.4 | 98.8 | 96.8 |
| | % LMW | 0.0 | 0.0 | 0.0 | 0.0 | 0.0 | 0.2 | 0.6 |
| Charge | % Acidic | 14.0 | 15.2 | 18.8 | 23.2 | 27.4 | 37.6 | TD |
| Variant | % Main | 39.1 | 37.4 | 30.7 | 26.4 | 23.9 | 17.3 | TD |
| Analysis by | % Basic | 46.9 | 47.4 | 50.5 | 50.4 | 48.7 | 45.2 | TD |
| CEX-UPLC | | | | | | | | |
| Charge | % Acidic | 20.3 | NR | NR | 27.1 | NR | NR | TD |
| Variant | % Main | 70.8 | NR | NR | 59.0 | NR | NR | TD |
| Analysis by | % Basic | 8.9 | NR | NR | 13.9 | NR | NR | TD |
| iCIEF | | | | | | | | |

CEX, cation exchange; DS, drug substance; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 9
Research Stability of REGN1979 Drug Product-Effect of Accelerated
Conditions
Formulation 2 mg/mL REGN1979, 10 mM histidine, 10% (w/v) sucrose,
0.1% (w/v) polysorbate 80, pH 5.8 (with nitrogen overlay)
Fill Volume 5.5 mL
Container/Closure 10 mL Type 1 borosilicate glass vials with a 20 mm FluroTec ®
coated West V10W-F597W 4432/50 GRY B2-TR stoppers

| | | | 25° C./60% RH Storage | | | | | |
|---|---|---|---|---|---|---|---|---|
| | | | (months) | | | 45° C. Storage (months) | | |
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 | | 0.00 | 0.00 | 0.00 | 0.00 | 0.00 | 0.00 | 0.01 |
| nm) | | | | | | | | |
| pH | | 5.8 | 5.8 | 5.8 | 5.9 | 5.8 | 5.8 | 5.8 |
| Particulate | 2-10 μm | 299 | NR | NR | 351 | NR | NR | 881 |
| Analysis by | ≥10 μm | 9 | NR | NR | 11 | NR | NR | 21 |
| MFI | ≥25 μm | 3 | NR | NR | 5 | NR | NR | 3 |
| (particles/mL) | | | | | | | | |
| % Protein Recovered by RP- | | 100 | 104 | 101 | 100 | 99 | 104 | 100 |
| UPLC | | | | | | | | |
| Purity by | Non-reduced; | 96.8 | NR | NR | 95.5 | NR | NR | 93.6 |
| MCE | % main peak | | | | | | | |
| | Reduced; | 99.5 | NR | NR | 99.9 | NR | NR | 97.5 |
| | % heavy + light | | | | | | | |
| | chain | | | | | | | |
| Purity by | % HMW | 0.6 | 0.6 | 0.6 | 0.6 | 0.6 | 0.9 | 2.4 |
| SE-UPLC | % Native | 99.4 | 99.4 | 99.4 | 99.4 | 99.4 | 98.9 | 97.0 |
| | % LMW | 0.0 | 0.0 | 0.0 | 0.0 | 0.0 | 0.1 | 0.6 |
| Charge | % Acidic | 14.8 | 14.9 | 18.9 | 22.3 | 27.4 | 35.0 | TD |
| Variant | % Main | 38.5 | 38.6 | 31.9 | 27.7 | 24.2 | 19.4 | TD |
| Analysis by | % Basic | 46.8 | 46.5 | 49.2 | 50.0 | 48.5 | 45.6 | TD |
| CEX-UPLC | | | | | | | | |
| Charge | % Acidic | 21.8 | NR | NR | 27.9 | NR | NR | TD |
| Variant | % Main | 70.2 | NR | NR | 57.6 | NR | NR | TD |
| Analysis by | % Basic | 8.0 | NR | NR | 14.5 | NR | NR | TD |
| iCIEF | | | | | | | | |

CEX, cation exchange; DS, drug substance; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 10
Research Stability of REGN1979 Drug Product-Effect of Stress Conditions
Formulation 2 mg/mL REGN1979, 10 mM histidine, 10% (w/v) sucrose,
0.1% (w/v) polysorbate 80, pH 5.8 (with nitrogen overlay)
Fill Volume 1.2 mL
Container/Closure 2 mL Type 1 borosilicate glass vials with a 13 mm FluroTec ®
coated West V2-F451W 4432/50 GRY B2-TR stoppers

| | | | Agitation (minutes) | | Freeze/Thaw (cycles) | |
|---|---|---|---|---|---|---|
| Assay | | 0 | 60 | 120 | 4 | 8 |
| Color and Appearance | | Pass | Pass | Pass | Pass | Pass |
| Turbidity (Increase in OD at | | 0.00 | 0.00 | 0.00 | 0.00 | 0.00 |
| 405 nm) | | | | | | |
| pH | | 5.8 | 5.8 | 5.8 | 5.8 | 5.8 |
| Particulate | 2-10 μm | 272 | NR | 268 | NR | 10327 |
| Analysis by | ≥10 μm | 7 | NR | 29 | NR | 75 |
| MFI | ≥25 μm | 0 | NR | 21 | NR | 16 |
| (particles/mL) | | | | | | |
| % Protein Recovered by RP- | | 100 | 100 | 100 | 106 | 99 |
| UPLC | | | | | | |
| Purity by | Non-reduced; | 97.7 | NR | 96.8 | NR | 96.8 |
| MCE | % main peak | | | | | |
| | Reduced; | 99.7 | NR | 99.8 | NR | 99.9 |
| | % heavy + light | | | | | |
| | chain | | | | | |
| Purity by | % HMW | 0.6 | 0.7 | 0.7 | 0.8 | 0.7 |
| SE-UPLC | % Native | 99.4 | 99.3 | 99.4 | 99.2 | 99.3 |
| | % LMW | 0.0 | 0.0 | 0.0 | 0.0 | 0.0 |
| Charge | % Acidic | 14.0 | 13.4 | 13.6 | 13.5 | 13.6 |
| Variant | % Main | 39.1 | 35.1 | 35.0 | 35.9 | 35.3 |
| Analysis by | % Basic | 46.9 | 51.6 | 51.4 | 50.5 | 51.2 |
| CEX-UPLC | | | | | | |
| Charge | % Acidic | 20.3 | NR | 20.9 | NR | 20.6 |
| Variant | % Main | 70.8 | NR | 69.6 | NR | 69.6 |
| Analysis by | % Basic | 8.9 | NR | 10.0 | NR | 9.6 |
| iCIEF | | | | | | |

CEX, cation exchange; DS, drug substance; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 11
Research Stability of REGN1979 Drug Product-Effect of Stress Conditions
Formulation 2 mg/mL REGN1979, 10 mM histidine, 10% (w/v) sucrose,
0.1% (w/v) polysorbate 80, pH 5.8 (with nitrogen overlay)
Fill Volume 5.5 mL
Container/Closure 10 mL Type 1 borosilicate glass vials with a 20 mm FluroTec ®
coated West V10W-F597W 4432/50 GRY B2-TR stoppers

| | | | Agitation (minutes) | | Freeze/Thaw (cycles) | |
|---|---|---|---|---|---|---|
| Assay | | 0 | 60 | 120 | 4 | 8 |
| Color and Appearance | | Pass | Pass | Pass | Pass | Pass |
| Turbidity (Increase in OD at | | 0.00 | 0.00 | 0.00 | 0.00 | 0.00 |
| 405 nm) | | | | | | |
| pH | | 5.8 | 5.8 | 5.8 | 5.8 | 5.8 |
| Particulate | 2-10 μm | 299 | NR | 3742 | NR | 5606 |
| Analysis by | ≥10 μm | 9 | NR | 21 | NR | 18 |
| MFI | ≥25 μm | 3 | NR | 0 | NR | 0 |
| (particles/mL) | | | | | | |
| % Protein Recovered by RP- | | 100 | 100 | 100 | 100 | 100 |
| UPLC | | | | | | |
| Purity by | Non-reduced; | 96.8 | NR | 97.8 | NR | 97.2 |
| MCE | % main peak | | | | | |
| | Reduced; | 99.5 | NR | 99.4 | NR | 99.7 |
| | % heavy + light | | | | | |
| | chain | | | | | |
| Purity by | % HMW | 0.6 | 0.6 | 0.6 | 0.6 | 0.6 |
| SE-UPLC | % Native | 99.4 | 99.4 | 99.4 | 99.4 | 99.4 |
| | % LMW | 0.0 | 0.0 | 0.0 | 0.0 | 0.0 |
| Charge | % Acidic | 14.8 | 12.6 | 13.3 | 12.6 | 12.8 |
| Variant | % Main | 38.5 | 36.7 | 36.1 | 36.7 | 36.4 |
| Analysis by | % Basic | 46.8 | 50.8 | 50.6 | 50.7 | 50.8 |
| CEX-UPLC | | | | | | |
| Charge | % Acidic | 21.8 | NR | 21.0 | NR | 21.9 |
| Variant | % Main | 70.2 | NR | 69.8 | NR | 68.8 |
| Analysis by | % Basic | 8.0 | NR | 9.2 | NR | 9.3 |
| iCIEF | | | | | | |

CEX, cation exchange; DS, drug substance; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

The results from the storage, accelerated, and stress stability studies indicate that REGN1979 will be stable during manufacture (formulation, fill/finish, and labeling operations) and can withstand short exposures to room temperature without compromising physical or chemical stability.

Additional stability experiments were conducted for formulations containing 2 mg/ml, 20 mg/ml, 100 mg/ml, and 160 mg/ml of the bispecific antibody (REGN1979) with 10 mM histidine, 10% w/v sucrose, 0.1% w/v polysorbate 80 and pH 5.8. These results are shown in Tables 12 to 27, below. NR=not reported; ND=too degraded to be analyzed.

TABLE 12
Research Stability of 2 mg/mL REGN1979 Drug Product (5% headspace O2)
Stored at 5° C.
Formulation 2 mg/mL REGN1979, 10 mM histidine, 10% (w/v) sucrose, 0.1%
(w/v) polysorbate 80, pH 5.8, 5% headspace O2
Fill Volume 1.2 mL
Container/Closure 2 mL Type 1 borosilicate glass vials with 13 mm FluroTec ® coated
West V2-F451W 4432/50 GRY B2-TR stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | | | |
| Turbidity (Increase in OD at 405 nm) | | 0.00 | 0.00 | 0.00 | 0.00 | 0.01 | 0.00 | | | |
| pH | | 5.8 | 5.9 | 5.9 | 5.8 | 5.8 | 5.8 | | | |
| Particulate | ≥10 μm | 6 | NR | NR | 11 | NR | 21 | | | |
| analysis by | ≥25 μm | 1 | NR | NR | 0 | NR | 1 | | | |
| HIAC | | | | | | | | | | |
| (particles/mL) | | | | | | | | | | |
| Particulate | 2-10 μm | 146 | NR | NR | 847 | NR | 1076 | | | |
| analysis by | ≥10 μm | 4 | NR | NR | 23 | NR | 8 | | | |
| MFI | ≥25 μm | 0 | NR | NR | 4 | NR | 2 | | | |
| (particles/mL) | | | | | | | | | | |
| % Protein Recovered by RP-UPLC | | 100 | 102 | 100 | 103 | 108 | 106 | | | |
| Purity by | Non-reduced; | 97.2 | NR | NR | 97.0 | NR | 97.2 | | | |
| MCE | % main peak | | | | | | | | | |
| | Reduced; | 98.3 | NR | NR | 97.9 | NR | 98.5 | | | |
| | % heavy + light | | | | | | | | | |
| | chain | | | | | | | | | |
| Purity by | % HMW | 0.8 | 0.8 | 0.8 | 0.8 | 0.8 | 0.7 | | | |
| SE-UPLC | % Native | 99.1 | 99.1 | 99.1 | 99.2 | 99.2 | 99.2 | | | |
| | % LMW | 0.1 | 0.1 | 0.1 | 0.0 | 0.0 | 0.0 | | | |
| Charge Variant | % Acidic | 13.8 | 13.2 | 13.4 | 12.7 | 13.2 | 14.5 | | | |
| Analysis by | % Main | 37.3 | 36.7 | 36.4 | 36.3 | 36.4 | 36.9 | | | |
| CEX-UPLC | % Basic | 49.0 | 50.2 | 50.2 | 50.9 | 50.4 | 48.6 | | | |
| Charge Variant | % Acidic | 24.2 | NR | NR | 25.2 | NR | 27.0 | | | |
| Analysis by | % Main | 61.7 | NR | NR | 59.8 | NR | 59.2 | | | |
| iCIEF | % Basic | 14.1 | NR | NR | 15.0 | NR | 13.8 | | | |

| | | | | | | |
|---|---|---|---|---|---|---|
| % Relative Potency by Bioassay | 131 | NR | NR | 103 | NR | 98 |

CEX, cation exchange; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 13
Research Stability of 2 mg/mL REGN1979 Drug Product (5% headspace O2)-
Effect of Accelerated Conditions
Formulation 2 mg/mL REGN1979, 10 mM histidine, 10% (w/v) sucrose, 0.1% (w/v)
polysorbate 80, pH 5.8, 5% headspace O2
Fill Volume 1.2 mL
Container/Closure 2 mL Type 1 borosilicate glass vials with 13 mm FluroTec ® coated West
V2-F451W 4432/50 GRY B2-TR stoppers

| | | | 25° C./60% RH Storage (months) | | | 45° C. Storage (months) | | |
|---|---|---|---|---|---|---|---|---|
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 nm) | | 0.00 | 0.00 | 0.00 | 0.00 | 0.00 | 0.00 | 0.01 |
| pH | | 5.8 | 5.9 | 5.9 | 5.8 | 5.9 | 5.9 | 5.9 |
| Particulate | ≥10 μm | 6 | NR | NR | 6 | NR | NR | 13 |
| analysis by HIAC | ≥25 μm | 1 | NR | NR | 0 | NR | NR | 1 |
| (particles/mL) | | | | | | | | |
| Particulate | 2-10 μm | 146 | NR | NR | 2354 | NR | NR | 522 |
| analysis by MFI | ≥10 μm | 4 | NR | NR | 23 | NR | NR | 29 |
| (particles/mL) | ≥25 μm | 0 | NR | NR | 10 | NR | NR | 0 |
| % Protein Recovered by RP-UPLC | | 100 | 101 | 100 | 103 | 99 | 101 | 99 |
| Purity by | Non-reduced; | 97.2 | NR | NR | 95.6 | NR | NR | 89.9 |
| MCE | % main peak | | | | | | | |
| | Reduced; | 98.3 | NR | NR | 98.5 | NR | NR | 97.7 |
| | % heavy + | | | | | | | |
| | light chain | | | | | | | |
| Purity by | % HMW | 0.8 | 0.8 | 0.8 | 0.8 | 1.2 | 1.8 | 9.0 |
| SE-UPLC | % Native | 99.1 | 99.2 | 99.1 | 99.2 | 98.6 | 98.0 | 90.3 |
| | % LMW | 0.1 | 0.1 | 0.1 | 0.0 | 0.2 | 0.2 | 0.7 |
| Charge Variant | % Acidic | 13.8 | 12.8 | 14.2 | 22.4 | 27.7 | 38.8 | TD |
| Analysis by | % Main | 37.3 | 36.5 | 36.0 | 27.2 | 26.4 | 14.9 | TD |
| CEX-UPLC | % Basic | 49.0 | 50.7 | 49.7 | 50.4 | 45.9 | 46.2 | TD |
| Charge Variant | % Acidic | 24.2 | NR | NR | 30.0 | NR | NR | TD |
| Analysis by iCIEF | % Main | 61.7 | NR | NR | 49.4 | NR | NR | TD |
| | % Basic | 14.1 | NR | NR | 20.6 | NR | NR | TD |

CEX, cation exchange; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, TD, too degraded to analyze; ultra-performance liquid chromatography

TABLE 14
Research Stability of 2 mg/mL REGN1979 Drug Product (5% headspace O2)-
Effect of Stress Conditions
Formulation 2 mg/mL REGN1979, 10 mM histidine, 10% (w/v) sucrose, 0.1% (w/v)
polysorbate 80, pH 5.8 (with nitrogen overlay)
Fill Volume 1.2 mL
Container/Closure 2 mL Type 1 borosilicate glass vials with 13 mm FluroTec ® coated
West V2-F451W 4432/50 GRY B2-TR stoppers

| | | | Agitation (hours) | | Freeze/Thaw (cycles) | |
|---|---|---|---|---|---|---|
| Assay | | 0 | 24 | 48 | 4 | 8 |
| Color and Appearance | | Pass | Pass | Pass | Pass | Pass |
| Turbidity (Increase in OD at 405 nm) | | 0.00 | 0.00 | 0.00 | 0.00 | 0.00 |
| pH | | 5.8 | 5.8 | 5.8 | 5.8 | 5.9 |
| Particulate | ≥10 μm | 6 | NR | 2 | NR | NR |
| analysis by HIAC | ≥25 μm | 1 | NR | 1 | NR | NR |
| (particles/mL) | | | | | | |
| Particulate | 2-10 μm | 146 | NR | 165 | NR | 1625 |
| analysis by MFI | ≥10 μm | 4 | NR | 2 | NR | 25 |
| (particles/mL) | ≥25 μm | 0 | NR | 0 | NR | 4 |
| % Protein Recovered by RP-UPLC | | 100 | 100 | 100 | 101 | 101 |
| Purity by | Non-reduced; | 97.2 | NR | 97.3 | NR | 97.1 |
| MCE | % main peak | | | | | |
| | Reduced; | 98.3 | NR | 98.5 | NR | 98.5 |
| | % heavy + | | | | | |
| | light chain | | | | | |
| Purity by | % HMW | 0.8 | 0.8 | 0.8 | 0.8 | 0.8 |
| SE-UPLC | % Native | 99.1 | 99.1 | 99.1 | 99.1 | 99.1 |
| | % LMW | 0.1 | 0.1 | 0.1 | 0.1 | 0.1 |
| Charge Variant | % Acidic | 13.8 | 13.5 | 13.9 | 13.4 | 13.0 |
| Analysis by | % Main | 37.3 | 37.4 | 37.7 | 35.5 | 36.5 |
| CEX-UPLC | % Basic | 49.0 | 49.1 | 48.4 | 51.1 | 50.4 |
| Charge Variant | % Acidic | 24.2 | NR | 24.0 | NR | 24.0 |
| Analysis by iCIEF | % Main | 61.7 | NR | 61.2 | NR | 61.9 |
| | % Basic | 14.1 | NR | 14.8 | NR | 14.1 |

CEX, cation exchange; HMW, high molecular weight; iCIEF imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 15
Research Stability of 20 mg/mL REGN1979 Drug Product Stored at 5° C.-
4.5 mL Fill in 10 mL Glass Vial
Formulation 20 mg/mL REGN1979, 10 mM histidine, 10% (w/v) sucrose, 0.1% (w/v)
polysorbate 80, pH 5.8
Fill Volume 4.5 mL
Container/Closure 10 mL Type 1 borosilicate glass vials with a 20 mm FluroTec ® coated West
S10-F451 4432/50 GRY B2-40 stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | | | |
| Turbidity (Increase in OD at 405 nm) | | 0.00 | 0.00 | 0.00 | 0.00 | 0.00 | 0.00 | | | |
| pH | | 5.8 | 5.8 | 5.8 | 5.9 | 5.8 | 5.8 | | | |
| Particulate | ≥10 μm | 3 | NR | NR | 16 | NR | 3 | | | |
| analysis by HIAC | ≥25 μm | 0 | NR | NR | 1 | NR | 1 | | | |
| (particles/mL) | | | | | | | | | | |
| Particulate | 2-10 μm | 293 | NR | NR | 3253 | NR | 754 | | | |
| analysis by | ≥10 μm | 3 | NR | NR | 10 | NR | 2 | | | |
| MFI | ≥25 μm | 0 | NR | NR | 2 | NR | 0 | | | |
| (particles/mL) | | | | | | | | | | |
| % Protein Recovered by RP-UPLC | | 100 | 102 | 99 | 96 | 96 | 105 | | | |
| Purity by | Non-reduced; | 96.5 | NR | NR | 96.4 | NR | 96.9 | | | |
| MCE | % main peak | | | | | | | | | |
| | Reduced; | 98.2 | NR | NR | 98.1 | NR | 98.0 | | | |
| | % heavy + | | | | | | | | | |
| | light chain | | | | | | | | | |
| Purity by | % HMW | 1.2 | 1.1 | 1.2 | 1.2 | 1.2 | 1.2 | | | |
| SE-UPLC | % Native | 98.1 | 98.5 | 98.4 | 98.3 | 98.5 | 98.1 | | | |
| | % LMW | 0.7 | 0.4 | 0.4 | 0.6 | 0.3 | 0.7 | | | |
| Charge Variant | % Acidic | 15.1 | 15.3 | 16.6 | 17.8 | 16.8 | 15.8 | | | |
| Analysis by | % Main | 33.3 | 33.4 | 29.6 | 32.2 | 30.1 | 30.8 | | | |
| CEX-UPLC | % Basic | 51.6 | 51.3 | 53.8 | 49.9 | 53.2 | 53.3 | | | |
| Charge Variant | % Acidic | 23.8 | NR | NR | 23.8 | NR | 26.2 | | | |
| Analysis by iCIEF | % Main | 61.6 | NR | NR | 61.6 | NR | 59.1 | | | |
| | % Basic | 14.7 | NR | NR | 14.6 | NR | 14.7 | | | |

| | | | | | | |
|---|---|---|---|---|---|---|
| % Relative Potency by Bioassay | 133 | NR | NR | 106 | NR | 120 |

CEX, cation exchange; HMW, high molecular weight; iCIEF, imaged capillary isoelectric focusing; LMW low molecular weight; MCE, microchip capillary electrophoresis; MFI, microflow imaging; NR, not required; OD, optica density; RH, relative humidity; RP, reversed-phase; SE, size exclusion; UPLC, ultra-performance liquid chromatography

TABLE 16
Research Stability of 20 mg/mL REGN1979 Drug Product-4.5 mL Fill in 10 mL Glass Vial-
Effect of Accelerated Conditions
Formulation 20 mg/mL REGN1979, 10 mM histidine, 10% (w/v) sucrose, 0.1% (w/v)
polysorbate 80, pH 5.8
Fill Volume 4.5 mL
Container/Closure 10 mL Type 1 borosilicate glass vials with a 20 mm FluroTec ® coated West
S10-F451 4432/50 GRY B2-40 stoppers

| | | | 25° C./60% RH Storage (months) | | | 45° C. Storage (months) | | |
|---|---|---|---|---|---|---|---|---|
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 nm) | | 0.00 | 0.00 | 0.01 | 0.00 | 0.00 | 0.00 | 0.03 |
| pH | | 5.8 | 5.9 | 5.9 | 5.8 | 5.9 | 5.9 | 5.8 |
| Particulate | ≥10 μm | 3 | NR | NR | 4 | NR | NR | 28 |
| analysis by HIAC | ≥25 μm | 0 | NR | NR | 1 | NR | NR | 1 |
| (particles/mL) | | | | | | | | |
| Particulate | 2-10 μm | 293 | NR | NR | 2900 | NR | NR | 3645 |
| analysis by | | | | | | | | |
| MFI | ≥10 μm | 3 | NR | NR | 10 | NR | NR | 42 |
| (particles/mL) | ≥25 μm | 0 | NR | NR | 0 | NR | NR | 0 |
| % Protein Recovered by RP-UPLC | | 100 | 103 | 100 | 95 | 101 | 102 | 99 |
| Purity by | Non-reduced; | 96.5 | NR | NR | 96.2 | NR | NR | 90.2 |
| MCE | % main peak | | | | | | | |
| | Reduced; | 98.2 | NR | NR | 97.8 | NR | NR | 91.5 |
| | % heavy + | | | | | | | |
| | light chain | | | | | | | |
| Purity by | % HMW | 1.2 | 1.1 | 1.2 | 1.3 | 1.7 | 2.8 | 9.9 |
| SE-UPLC | % Native | 98.1 | 98.5 | 98.5 | 98.0 | 97.9 | 96.4 | 88.3 |
| | % LMW | 0.7 | 0.4 | 0.3 | 0.8 | 0.4 | 0.8 | 1.7 |
| Charge Variant | % Acidic | 15.1 | 16.2 | 19.8 | 22.5 | 25.8 | 39 | TD |
| Analysis by | % Main | 33.3 | 32.1 | 26.3 | 27.7 | 20.7 | 13.5 | TD |
| CEX-UPLC | % Basic | 51.6 | 51.7 | 53.9 | 49.8 | 53.5 | 47.5 | TD |
| Charge Variant | % Acidic | 23.8 | NR | NR | 29.3 | NR | NR | TD |
| Analysis by iCIEF | % Main | 61.6 | NR | NR | 50.6 | NR | NR | TD |
| | % Basic | 14.7 | NR | NR | 20.1 | NR | NR | TD |

CEX, cation exchange; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; TD, too degraded; UPLC, ultra-performance liquid chromatography

TABLE 17
Research Stability of 20 mg/mL REGN1979 Drug Product-4.5 mL Fill in 10 mL Glass Vial-
Effect of Stress Conditions
Formulation 20 mg/mL REGN1979, 10 mM histidine, 10% (w/v) sucrose, 0.1% (w/v)
polysorbate 80, pH 5.8
Fill Volume 4.5 mL
Container/Closure 10 mL Type 1 borosilicate glass vials with 20 mm FluroTec ® coated
West S10-F451 4432/50 GRY B2-40 stoppers

| | | | Agitation (hours) | | Freeze/Thaw (cycles) | |
|---|---|---|---|---|---|---|
| Assay | | 0 | 24 | 48 | 4 | 8 |
| Color and Appearance | | Pass | Pass | Pass | Pass | Pass |
| Turbidity (Increase in OD at 405 nm) | | 0.00 | 0.00 | 0.00 | 0.00 | 0.00 |
| pH | | 5.8 | 5.8 | 5.9 | 5.9 | 5.9 |
| Particulate | ≥10 μm | 3 | NR | 33 | NR | NR |
| analysis by HIAC | ≥25 μm | 0 | NR | 1 | NR | NR |
| (particles/mL) | | | | | | |
| Particulate | 2-10 μm | 293 | NR | 3580 | NR | 5563 |
| analysis by MFI | ≥10 μm | 3 | NR | 46 | NR | 15 |
| (particles/mL) | ≥25 μm | 0 | NR | 3 | NR | 3 |
| % Protein Recovered by RP-UPLC | | 100 | 99 | 100 | 98 | 99 |
| Purity by | Non-reduced; | 96.5 | NR | 96.7 | NR | 96.8 |
| MCE | % main peak | | | | | |
| | Reduced; | 98.2 | NR | 98.2 | NR | 98.2 |
| | % heavy + | | | | | |
| | light chain | | | | | |
| Purity by | % HMW | 1.2 | 1.1 | 1.1 | 1.2 | 1.2 |
| SE-UPLC | % Native | 98.1 | 98.3 | 98.4 | 98.3 | 98.4 |
| | % LMW | 0.7 | 0.6 | 0.5 | 0.4 | 0.4 |
| Charge Variant | % Acidic | 15.1 | 13.4 | 15.2 | 15.6 | 15.3 |
| Analysis by | % Main | 33.3 | 32.5 | 30.6 | 33.5 | 33.2 |
| CEX-UPLC | % Basic | 51.6 | 54.1 | 54.2 | 50.9 | 51.5 |
| Charge Variant | % Acidic | 23.8 | NR | 23.7 | NR | 23.3 |
| Analysis by iCIEF | % Main | 61.1 | NR | 61.6 | NR | 62.5 |
| | % Basic | 14.7 | NR | 14.7 | NR | 14.2 |

CEX, cation exchange; HMW, high molecular weight; iCIEF imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 18
Research Stability of 160 mg/mL REGN1979 Drug Product Stored at 5° C.-
8.5 mL Fill in 10 mL Glass Vial
Formulation 20 mg/mL REGN1979, 10 mM histidine, 10% (w/v) sucrose, 0.1% (w/v)
polysorbate 80, pH 5.8
Fill Volume 8.5 mL
Container/Closure 10 mL Type 1 borosilicate glass vials with a 20 mm FluroTec ® coated West
S10-F451 4432/50 GRY B2-40 stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | | | |
| Turbidity (Increase in OD at 405 nm) | | 0.00 | 0.00 | 0.00 | 0.00 | 0.00 | 0.00 | | | |
| pH | | 5.8 | 5.8 | 5.8 | 5.9 | 5.8 | 5.8 | | | |
| Particulate | ≥10 μm | 1 | NR | NR | 4 | NR | 4 | | | |
| analysis by HIAC | ≥25 μm | 0 | NR | NR | 0 | NR | 0 | | | |
| (particles/mL) | | | | | | | | | | |
| Particulate | 2-10 μm | 274 | NR | NR | 1059 | NR | 690 | | | |
| analysis by MFI | ≥10 μm | 34 | NR | NR | 0 | NR | 2 | | | |
| (particles/mL) | ≥25 μm | 5 | NR | NR | 0 | NR | 2 | | | |
| % Protein Recovered by RP-UPLC | | 100 | 102 | 99 | 95 | 96 | 102 | | | |
| Purity by | Non-reduced; | 97.0 | NR | NR | 97.0 | NR | 97.2 | | | |
| MCE | % main peak | | | | | | | | | |
| | Reduced; | 97.8 | NR | NR | 97.0 | NR | 97.6 | | | |
| | % heavy + | | | | | | | | | |
| | light chain | | | | | | | | | |
| Purity by | % HMW | 1.2 | 1.1 | 1.2 | 1.2 | 1.2 | 1.2 | | | |
| SE-UPLC | % Native | 98.2 | 98.5 | 98.5 | 98.2 | 98.5 | 98.4 | | | |
| | % LMW | 0.6 | 0.4 | 0.4 | 0.6 | 0.3 | 0.5 | | | |
| Charge Variant | % Acidic | 15.4 | 15.0 | 14.5 | 14.8 | 16.2 | 15.4 | | | |
| Analysis by | % Main | 34.4 | 34.1 | 33.9 | 34.3 | 29.2 | 32.4 | | | |
| CEX-UPLC | % Basic | 50.2 | 50.9 | 51.7 | 50.9 | 54.6 | 52.2 | | | |
| Charge Variant | % Acidic | 24.0 | NR | NR | 29.3 | NR | 26.2 | | | |
| Analysis by iCIEF | % Main | 61.6 | NR | NR | 51.0 | NR | 60.0 | | | |
| | % Basic | 14.4 | NR | NR | 19.7 | NR | 13.9 | | | |

| | | | | | | |
|---|---|---|---|---|---|---|
| % Relative Potency by Bioassay | 115 | NR | NR | 103 | NR | 119 |

CEX, cation exchange; HMW, high molecular weight; iCIEF, imaged capillary isoelectric focusing; LMW low molecular weight; MCE, microchip capillary electrophoresis; MFI, microflow imaging; NR, not required; OD, optica density; RH, relative humidity; RP, reversed-phase; SE, size exclusion; UPLC, ultra-performance liquid chromatography

TABLE 19
Research Stability of 160 mg/mL REGN1979 Drug Product-8.5 mL Fill in 10 mL Glass Vial-
Effect of Accelerated Conditions
Formulation 20 mg/mL REGN1979, 10 mM histidine, 10% (w/v) sucrose, 0.1% (w/v)
polysorbate 80, pH 5.8
Fill Volume 8.5 mL
Container/Closure 10 mL Type 1 borosilicate glass vials with a 20 mm FluroTec ® coated West
S10-F451 4432/50 GRY B2-40 stoppers

| | | | 25° C./60% RH Storage (months) | | | 45° C. Storage (months) | | |
|---|---|---|---|---|---|---|---|---|
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 nm) | | 0.00 | 0.01 | 0.01 | 0.00 | 0.00 | 0.00 | 0.03 |
| pH | | 5.8 | 5.9 | 5.9 | 5.8 | 5.9 | 5.9 | 5.8 |
| Particulate | ≥10 μm | 1 | NR | NR | 3 | NR | NR | 23 |
| analysis by HIAC | ≥25 μm | 0 | NR | NR | 1 | NR | NR | 1 |
| (particles/mL) | | | | | | | | |
| Particulate | 2-10 μm | 293 | NR | NR | 2900 | NR | NR | 3645 |
| analysis by | ≥10 μm | 3 | NR | NR | 10 | NR | NR | 42 |
| MFI | ≥25 μm | 0 | NR | NR | 0 | NR | NR | 0 |
| (particles/mL) | | | | | | | | |
| % Protein Recovered by RP-UPLC | | 100 | 103 | 100 | 95 | 101 | 102 | 99 |
| Purity by | Non-reduced; | 96.5 | NR | NR | 96.2 | NR | NR | 90.2 |
| MCE | % main peak | | | | | | | |
| | Reduced; | 98.2 | NR | NR | 97.8 | NR | NR | 91.5 |
| | % heavy + | | | | | | | |
| | light chain | | | | | | | |
| Purity by | % HMW | 1.2 | 1.1 | 1.2 | 1.3 | 1.7 | 2.8 | 9.9 |
| SE-UPLC | % Native | 98.1 | 98.5 | 98.5 | 98.0 | 97.9 | 96.4 | 88.3 |
| | % LMW | 0.7 | 0.4 | 0.3 | 0.8 | 0.4 | 0.8 | 1.7 |
| Charge Variant | % Acidic | 15.1 | 16.2 | 19.8 | 22.5 | 25.8 | 39 | TD |
| Analysis by | % Main | 33.3 | 32.1 | 26.3 | 27.7 | 20.7 | 13.5 | TD |
| CEX-UPLC | % Basic | 51.6 | 51.7 | 53.9 | 49.8 | 53.5 | 47.5 | TD |
| Charge Variant | % Acidic | 23.8 | NR | NR | 29.3 | NR | NR | TD |
| Analysis by | % Main | 61.6 | NR | NR | 50.6 | NR | NR | TD |
| iCIEF | % Basic | 14.7 | NR | NR | 20.1 | NR | NR | TD |

| | | | | | | | |
|---|---|---|---|---|---|---|---|
| % Relative Potency by Bioassay | 14.4 | NR | NR | 14.6 | NR | NR | TD |

CEX, cation exchange; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; TD, too degraded; UPLC, ultra-performance liquid chromatography

TABLE 20
Research Stability of 160 mg/mL REGN1979 Drug Product-8.5 mL Fill in 10 mL Glass Vial-
Effect of Stress Conditions
Formulation 20 mg/mL REGN1979, 10 mM histidine, 10% (w/v) sucrose, 0.1% (w/v)
polysorbate 80, pH 5.8
Fill Volume 8.5 mL
Container/Closure 10 mL Type 1 borosilicate glass vials with 20 mm FluroTec ® coated
West S10-F451 4432/50 GRY B2-40 stoppers

| | | | Agitation (hours) | | Freeze/Thaw (cycles) | |
|---|---|---|---|---|---|---|
| Assay | | 0 | 24 | 48 | 4 | 8 |
| Color and Appearance | | Pass | Pass | Pass | Pass | Pass |
| Turbidity (Increase in OD at 405 nm) | | 0.00 | 0.00 | 0.00 | 0.00 | 0.00 |
| pH | | 5.8 | 5.8 | 5.9 | 5.9 | 5.9 |
| Particulate | ≥10 μm | 1 | NR | 13 | NR | NR |
| analysis by HIAC | ≥25 μm | 0 | NR | 1 | NR | NR |
| (particles/mL) | | | | | | |
| Particulate | 2-10 μm | 724 | NR | 1658 | NR | 2340 |
| analysis by MFI | ≥10 μm | 34 | NR | 9 | NR | 5 |
| (particles/mL) | ≥25 μm | 5 | NR | 0 | NR | 30 |
| % Protein Recovered by RP-UPLC | | 100 | 99 | 103 | 100 | 99 |
| Purity by | Non-reduced; | 97.0 | NR | 97.0 | NR | 97.0 |
| MCE | % main peak | | | | | |
| | Reduced; | 97.8 | NR | 97.9 | NR | 98.1 |
| | % heavy + | | | | | |
| | light chain | | | | | |
| Purity by | % HMW | 1.2 | 1.1 | 1.1 | 1.2 | 1.2 |
| SE-UPLC | % Native | 98.2 | 98.4 | 98.4 | 98.3 | 98.3 |
| | % LMW | 0.6 | 0.5 | 0.5 | 0.4 | 0.5 |
| Charge Variant | % Acidic | 15.4 | 14.3 | 14.8 | 15.1 | 15.4 |
| Analysis by | % Main | 34.4 | 33.8 | 34.0 | 34.5 | 33.9 |
| CEX-UPLC | % Basic | 50.2 | 51.9 | 51.1 | 50.4 | 50.7 |
| Charge Variant | % Acidic | 24.0 | NR | 25.1 | NR | 24.3 |
| Analysis by iCIEF | % Main | 61.6 | NR | 60.2 | NR | 61.8 |
| | % Basic | 14.4 | NR | 14.7 | NR | 14.0 |

CEX, cation exchange; HMW, high molecular weight; iCIEF imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 21
Research Stability of 100 mg/mL REGN1979 Drug Product Stored at 5° C.
Formulation 100 mg/mL REGN1979, 10 mM histidine, 10% (w/v) sucrose, 0.1% (w/v)
polysorbate 80, pH 5.8
Fill Volume 2.5 mL
Container/Closure 5 mL Type 1 borosilicate glass vials with a 20 mm FluroTec ® coated West
S10-F451 4432/50 GRY B2-40 stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | | | |
| Turbidity (Increase in OD at 405 nm) | | 0.00 | 0.00 | 0.00 | 0.00 | 0.00 | 0.00 | | | |
| pH | | 5.9 | 5.8 | 5.9 | 5.8 | 5.9 | 5.8 | | | |
| Particulate | ≥10 μm | 4 | NR | NR | 59 | NR | 3 | | | |
| analysis by HIAC | ≥25 μm | 1 | NR | NR | 1 | NR | 1 | | | |
| (particles/mL) | | | | | | | | | | |
| Particulate | 2-10 μm | 3213 | NR | NR | 3524 | NR | 4539 | | | |
| analysis by MFI | ≥10 μm | 4 | NR | NR | 48 | NR | 15 | | | |
| (particles/mL) | ≥25 μm | 0 | NR | NR | 6 | NR | 0 | | | |
| % Protein Recovered by RP-UPLC | | 100 | 102 | 99 | 100 | 100 | 97 | | | |
| Purity by | Non-reduced; | 96.3 | NR | NR | 96.3 | NR | 96.4 | | | |
| MCE | % main peak | | | | | | | | | |
| | Reduced; | 98.3 | NR | NR | 98.4 | NR | 98.4 | | | |
| | % heavy + | | | | | | | | | |
| | light chain | | | | | | | | | |
| Purity by | % HMW | 1.3 | 1.3 | 1.4 | 1.4 | 1.4 | 1.5 | | | |
| SE-UPLC | % Native | 9.8 | 98.2 | 98.2 | 98.1 | 98.0 | 98.0 | | | |
| | % LMW | 0.4 | 0.4 | 0.4 | 0.4 | 0.6 | 0.5 | | | |
| Charge Variant | % Acidic | 11.5 | 11.2 | 11.5 | 10.7 | 11.7 | 12.0 | | | |
| Analysis by | % Main | 37.4 | 35.9 | 37.4 | 38.7 | 37.0 | 34.2 | | | |
| CEX-UPLC | % Basic | 51.1 | 52.9 | 51.1 | 50.7 | 51.2 | 53.8 | | | |
| Charge Variant | % Acidic | 23.8 | NR | NR | 23.8 | NR | 26.5 | | | |
| Analysis by iCIEF | % Main | 59.9 | NR | NR | 59.9 | NR | 58.6 | | | |
| | % Basic | 16.3 | NR | NR | 16.3 | NR | 15.0 | | | |

| | | | | | | |
|---|---|---|---|---|---|---|
| % Relative Potency by Bioassay | 114 | NR | NR | 127 | NR | 102 |

CEX, cation exchange; HMW, high molecular weight; iCIEF, imaged capillary isoelectric focusing; LMW low molecular weight; MCE, microchip capillary electrophoresis; MFI, microflow imaging; NR, not required; OD, optica density; RH, relative humidity; RP, reversed-phase; SE, size exclusion; UPLC, ultra-performance liquid chromatography

TABLE 22
Research Stability of 100 mg/mL REGN1979 Drug Product-Effect of Accelerated Conditions
Formulation 100 mg/mL REGN1979, 10 mM histidine, 10% (w/v) sucrose, 0.1% (w/v)
polysorbate 80, pH 5.8
Fill Volume 2.5 mL
Container/Closure 5 mL Type 1 borosilicate glass vials with a 20 mm FluroTec ® coated West
S10-F451 4432/50 GRY B2-40 stoppers

| | | | 25° C./60% RH Storage (months) | | | 45° C. Storage (months) | | |
|---|---|---|---|---|---|---|---|---|
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 nm) | | 0.00 | 0.00 | 0.01 | 0.01 | 0.01 | 0.03 | 0.12 |
| pH | | 5.9 | 5.8 | 5.9 | 5.9 | 5.9 | 5.9 | 5.9 |
| Particulate | ≥10 μm | 4 | NR | NR | 18 | NR | NR | 11 |
| analysis by HIAC | ≥25 μm | 1 | NR | NR | 0 | NR | NR | 0 |
| (particles/mL) | | | | | | | | |
| Particulate | 2-10 μm | 3213 | NR | NR | 3524 | NR | NR | 3667 |
| analysis by | ≥10 μm | 4 | NR | NR | 21 | NR | NR | 92 |
| MFI | ≥25 μm | 0 | NR | NR | 6 | NR | NR | 2 |
| (particles/mL) | | | | | | | | |
| % Protein Recovered by RP-UPLC | | 100 | 102 | 100 | 100 | 100 | 103 | 95 |
| Purity by | Non-reduced; | 96.3 | NR | NR | 95.1 | NR | NR | 86.0 |
| MCE | % main peak | | | | | | | |
| | Reduced; | 98.3 | NR | NR | 97.9 | NR | NR | 91.5 |
| | % heavy + | | | | | | | |
| | light chain | | | | | | | |
| Purity by | % HMW | 1.3 | 1.5 | 1.7 | 1.9 | 3.3 | 5.6 | 20.3 |
| SE-UPLC | % Native | 98.3 | 98.1 | 97.9 | 97.4 | 96.0 | 93.4 | 78.2 |
| | % LMW | 0.4 | 0.4 | 0.4 | 0.8 | 0.7 | 1.0 | 1.5 |
| Charge Variant | % Acidic | 11.5 | 11.0 | 12.4 | 21.4 | 25.6 | 33.7 | TD |
| Analysis by | % Main | 37.4 | 35.8 | 36.1 | 27.8 | 24.6 | 16.1 | TD |
| CEX-UPLC | % Basic | 51.1 | 53.2 | 51.5 | 50.9 | 49.8 | 52.2 | TD |
| Charge Variant | % Acidic | 23.8 | NR | NR | 29.3 | NR | NR | TD |
| Analysis by | % Main | 59.9 | NR | NR | 48.3 | NR | NR | TD |
| iCIEF | % Basic | 16.3 | NR | NR | 22.4 | NR | NR | TD |

CEX, cation exchange; HMW, high molecular weight; iCIEF, imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; TD, too degraded; UPLC, ultra-performance liquid chromatography

TABLE 23
Research Stability of 100 mg/mL REGN1979 Drug Product-Effect of Stress Conditions
Formulation 100 mg/mL REGN1979, 10 mM histidine, 10% (w/v) sucrose, 0.1% (w/v)
polysorbate 80, pH 5.8
Fill Volume 2.5 mL
Container/Closure 5 mL Type 1 borosilicate glass vials with 20 mm FluroTec ® coated
West S10-F451 4432/50 GRY B2-40 stoppers

| | | | Agitation (hours) | | Freeze/Thaw (cycles) | |
|---|---|---|---|---|---|---|
| Assay | | 0 | 24 | 48 | 4 | 8 |
| Color and Appearance | | Pass | Pass | Pass | Pass | Pass |
| Turbidity (Increase in OD at 405 nm) | | 0.00 | 0.00 | 0.00 | 0.01 | 0.00 |
| pH | | 5.9 | 5.8 | 5.8 | 5.8 | 5.9 |
| Particulate | ≥10 μm | 4 | NR | 15 | NR | NR |
| analysis by HIAC | ≥25 μm | 1 | NR | 1 | NR | NR |
| (particles/mL) | | | | | | |
| Particulate | 2-10 μm | 3213 | NR | 202 | NR | 6318 |
| analysis by MFI | ≥10 μm | 4 | NR | 10 | NR | 8 |
| (particles/mL) | ≥25 μm | 0 | NR | 2 | NR | 0 |
| % Protein Recovered by RP-UPLC | | 100 | 101 | 102 | 102 | 101 |
| Purity by | Non-reduced; | 96.3 | NR | 96.3 | NR | 96.3 |
| MCE | % main peak | | | | | |
| | Reduced; | 98.3 | NR | 98.4 | NR | 98.4 |
| | % heavy + | | | | | |
| | light chain | | | | | |
| Purity by | % HMW | 1.3 | 1.3 | 1.4 | 1.4 | 1.4 |
| SE-UPLC | % Native | 98.3 | 98.3 | 98.3 | 98.1 | 98.1 |
| | % LMW | 0.4 | 0.4 | 0.4 | 0.5 | 0.5 |
| Charge Variant | % Acidic | 11.5 | 12.1 | 12.1 | 11.4 | 11.7 |
| Analysis by | % Main | 37.4 | 38.7 | 39.5 | 35.3 | 37.6 |
| CEX-UPLC | % Basic | 51.1 | 49.2 | 48.5 | 53.3 | 50.7 |
| Charge Variant | % Acidic | 23.8 | NR | 23.4 | NR | 24.1 |
| Analysis by iCIEF | % Main | 59.9 | NR | 59.9 | NR | 59.8 |
| | % Basic | 16.3 | NR | 16.7 | NR | 16.1 |

CEX, cation exchange; HMW, high molecular weight; iCIEF imaged capillary isoelectric focusing; LMW, low molecular weight; MCE, microchip capillary electrophoresis; MFI, microflow imaging; NR, not required; OD, optical density; RH, relative humidity; RP, reversed-phase; SE, size exclusion; UPLC, ultra-performance liquid chromatography

TABLE 24
Research Stability of 160 mg/mL REGN1979 Drug Product Stored at 5° C.
Formulation 160 mg/mL REGN1979, 10 mM histidine, 10% (w/v) sucrose, 0.1% (w/v)
polysorbate 80, pH 5.8
Fill Volume 1.2 mL
Container/Closure 2 mL Type 1 borosilicate glass vials with a 13 mm FluroTec ® coated West
S2 F451 4432/50 GRY B2-40 stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | Pass | | |
| Turbidity (Increase in OD at 405 nm) | | 0.00 | 0.014 | 0.00 | 0.01 | 0.01 | 0.07 | 0.00 | | |
| pH | | 5.7 | 5.9 | 5.8 | 5.9 | 5.9 | 5.7 | 5.8 | | |
| % Protein Recovered by RP-UPLC | | 100 | 99 | 96 | 98 | 94 | 94 | 95 | | |
| Purity by | % HMW | 1.1 | 1.3 | 1.3 | 1.4 | 1.5 | 1.1 | 1.6 | | |
| SE-UPLC | % Native | 98.8 | 98.4 | 98.6 | 98.4 | 98.3 | 98.4 | 98.1 | | |
| | % LMW | 0.1 | 0.4 | 0.1 | 0.2 | 0.2 | 0.6 | 0.2 | | |
| Charge Variant | % Acidic | 12.0 | 12.0 | 13.0 | 12.5 | 13.3 | 13.8 | 12.6 | | |
| Analysis by | % Main | 38.0 | 37.2 | 38.6 | 39.7 | 39.7 | 39.1 | 39.2 | | |
| CEX-UPLC | % Basic | 50.0 | 50.8 | 48.4 | 47.8 | 47.0 | 47.1 | 48.2 | | |

CEX, cation exchange; HMW, high molecular weight; LMW, low molecular weight; OD, optical density; RP, reversed-phase; SE, size exclusion; UPLC, ultra-performance liquid chromatography

TABLE 25
Research Stability of 160 mg/mL REGN1979 Drug Product Stored-Effect of Accelerated Conditions
Formulation 160 mg/mL REGN1979, 10 mM histidine, 10% (w/v) sucrose, 0.1% (w/v)
polysorbate 80, pH 5.8
Fill Volume 1.2 mL
Container/Closure 2 mL Type 1 borosilicate glass vials with a 13 mm FluroTec ® coated West
S2 F451 4432/50 B2 40 stoppers

| | | | 25° C./60% RH Storage (months) | | | 45° C. Storage (months) | | |
|---|---|---|---|---|---|---|---|---|
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 nm) | | 0.00 | 0.01 | 0.01 | 0.01 | 0.01 | 0.03 | 0.14 |
| pH | | 5.8 | 5.9 | 5.9 | 5.9 | 5.9 | 5.9 | 5.9 |
| % Protein Recovered by RP-UPLC | | 100 | 98 | 94 | 97 | 97 | 99 | 93 |
| Purity by | % HMW | 1.1 | 1.6 | 1.9 | 2.2 | 3.5 | 5.8 | 20.5 |
| SE-UPLC | % Native | 98.8 | 98.1 | 97.6 | 97.4 | 96.0 | 93.4 | 78.0 |
| | % LMW | 0.1 | 0.4 | 0.6 | 0.3 | 0.5 | 0.6 | 1.6 |
| Charge Variant | % Acidic | 12.0 | 11.8 | 14.8 | 16.6 | 22.5 | 34.5 | 49.6 |
| Analysis by | % Main | 38.0 | 36.6 | 36.9 | 35.9 | 24.2 | 17.6 | 15.5 |
| CEX-UPLC | % Basic | 50.0 | 51.5 | 48.3 | 47.5 | 53.3 | 47.9 | 35.0 |

CEX, cation exchange; HMW, high molecular weight; LMW, low molecular weight; OD, optical density; RP, reversed-phase; SE, size exclusion; UPLC, ultra-performance liquid chromatography

TABLE 26
Research Stability of 80 mg/mL REGN1979 Drug Product Stored at 5° C.
Formulation 80 mg/mL REGN1979, 10 mM histidine, 10% (w/v) sucrose, 0.1% (w/v)
polysorbate 80, pH 5.8
Fill Volume 2.5 mL
Container/Closure 5 mL Type 1 borosilicate glass vials with a 13 mm FluroTec ® coated West
S10 F451 4432/50 B2 40 stoppers

| | | Length of Storage at 5° C. (months) | | | | | | | | |
|---|---|---|---|---|---|---|---|---|---|---|
| Assay | | 0 | 1 | 3 | 6 | 9 | 12 | 18 | 24 | 36 |
| Color and Appearance | | Pass | Pass | Pass | Pass | Pass | Pass | Pass | | |
| Turbidity (Increase in OD at 405 nm) | | 0.00 | 0.00 | 0.00 | 0.00 | 0.00 | 0.00 | 0.00 | | |
| pH | | 5.8 | 5.8 | 5.8 | 5.8 | 5.8 | 5.8 | 5.8 | | |
| % Protein Recovered by RP-UPLC | | 100 | 101 | 103 | 99 | 97 | 99 | 98 | | |
| Purity by | % HMW | 1.1 | 1.1 | 1.0 | 1.1 | 1.2 | 1.2 | 1.3 | | |
| SE-UPLC | % Native | 98.9 | 86.6 | 98.8 | 98.5 | 98.6 | 98.5 | 98.6 | | |
| | % LMW | 0.1 | 0.3 | 0.2 | 0.3 | 0.2 | 0.3 | 0.2 | | |
| Charge Variant | % Acidic | 12.1 | 11.6 | 13.1 | 13.5 | 13.5 | 13.5 | 13.2 | | |
| Analysis by | % Main | 36.9 | 37.4 | 38.7 | 39.0 | 39.0 | 39.2 | 39.1 | | |
| CEX-UPLC | % Basic | 50.9 | 51.0 | 48.2 | 47.5 | 47.5 | 47.3 | 47.7 | | |

CEX, cation exchange; HMW, high molecular weight; LMW, low molecular weight; OD, optical density; RP, reversed-phase; SE, size exclusion; UPLC, ultra-performance liquid chromatography

TABLE 27
Research Stability of 80 mg/mL REGN1979 Drug Product Stored-Effect of Accelerated Conditions
Formulation 80 mg/mL REGN1979, 10 mM histidine, 10% (w/v) sucrose, 0.1% (w/v)
polysorbate 80, pH 5.8
Fill Volume 2.5 mL
Container/Closure 5 mL Type 1 borosilicate glass vials with a 13 mm FluroTec ® coated West
S10 F451 4432/50 B2 40 stoppers

| | | | 25° C./60% RH Storage (months) | | | 45° C. Storage (months) | | |
|---|---|---|---|---|---|---|---|---|
| Assay | | 0 | 1 | 3 | 6 | 0.5 | 1 | 3 |
| Color and Appearance | | Pass | Pass | Pass | Pass | Pass | Pass | Pass |
| Turbidity (Increase in OD at 405 nm) | | 0.00 | 0.00 | 0.01 | 0.01 | 0.01 | 0.02 | 0.08 |
| pH | | 5.8 | 5.8 | 5.8 | 5.8 | 5.8 | 5.8 | 5.8 |
| % Protein Recovered by RP-UPLC | | 100 | 99 | 103 | 100 | 101 | 100 | 98 |
| Purity by | % HMW | 1.0 | 1.2 | 1.4 | 1.5 | 2.3 | 3.7 | 14.7 |
| SE-UPLC | % Native | 98.9 | 98.4 | 98.3 | 98.1 | 97.3 | 95.5 | 83.5 |
| | % LMW | 0.1 | 0.4 | 0.4 | 0.4 | 0.4 | 0.6 | 1.6 |
| Charge Variant | % Acidic | 12.1 | 11.6 | 14.6 | 25.6 | 22.8 | 34.1 | 25.3 |
| Analysis by | % Main | 36.9 | 36.9 | 37.4 | 27.2 | 24.5 | 17.3 | 19.5 |
| CEX-UPLC | % Basic | 50.9 | 51.5 | 48.1 | 47.2 | 52.7 | 48.6 | 55.2 |

CEX, cation exchange; HMW, high molecular weight; LMW, low molecular weight; OD, optical density; RP, reversed-phase; SE, size exclusion; UPLC, ultra-performance liquid chromatography

Additional stability studies were conducted with various other excipients with a bispecific antibody (REGN1979) concentration of 100 mg/ml. The various formulations (F1-F13) are shown in Table 28, below. The results of these stability experiments are shown in Tables 29-41. In each of these experiments, the container/closure was a 5 mL Type 1 borosilicate glass vial with a 20 mm FluroTec® coated West V2-F451W 4432/50 GRY B2-TR stopper.

TABLE 28
Additional Formulations Evaluated

| Formulation # | Buffer | Surfactant | Stabilizer | pH |
|---|---|---|---|---|
| F1 | 10 mM histidine | 0.1% PS20 | 150 mM ArgHCl | 6.0 |
| F2 | 10 mM histidine | 0.1% PS20 | 5% sorbitol | 5.5 |
| F3 | 10 mM succinate | 0.1% PS20 | 150 mM NaCl | 6.0 |
| F4 | 10 mM acetate | 0.1% PS80 | 150 mM ArgHCl | 6.2 |
| F5 | 10 mM acetate | 0.1% | 5% sorbitol | 5.7 |
| | | Poloxamer 88 | | |
| F6 | 10 mM histidine | 0.1% PS80 | 150 mM NaCl | 5.5 |
| F7 | 10 mM histidine | 0.1% PS80 | 5% sorbitol | 6.0 |
| F8 | 10 mM succinate | 0.1% | 150 mM ArgHCl | 5.5 |
| | | Poloxamer 88 | | |
| F9 | 10 mM succinate | 0.1% PS20 | 5% sorbitol | 6.5 |
| F10 | 10 mM acetate | 0.1% | 150 mM NaCl | 6.2 |
| | | Poloxamer 88 | | |
| F11 | 10 mM acetate | 0.1% PS20 | 10% sucrose | 5.2 |
| F12 | 10 mM histidine | 0.1% | 10% sucrose | 6.5 |
| | | Poloxamer 88 | | |
| F13 | 10 mM succinate | 0.1% PS80 | 10% sucrose | 6.0 |

TABLE 29
Research Stability of Formulation Fl Stored at 45° C.

| Formulation | | 100 mg/mL REGN1979, 10 mM | | |
|---|---|---|---|---|
| | | histidine, 150 mM Arginine HCl, | | |
| | | 0.1% (w/v) polysorbate 20, pH 6.0 | | |
| Fill Volume | | 2.5 | | |
| | | Length of Storage at 45° C. (month) | | |
| Assay | | 0 | 0.5 | 1 |
| Color and Appearance | | Pass | Pass | Fail |
| Turbidity (Increase in OD at 405 nm) | | 0.00 | 0.04 | 0.10 |
| pH | | 6.1 | 6.1 | 6.2 |
| Particulate | 2-10 μm | 101 | NR | 563 |
| Analysis by | ≥10 μm | 5 | NR | 15 |
| MFI (particles/mL) | ≥25 μm | 0 | NR | 3 |
| % Protein Recovered by RP-UPLC | | 100 | 98 | 100 |
| Purity by SE-UPLC | % HMW | 2.5 | 8.2 | 14.8 |
| | % Native | 97.2 | 91.1 | 84.2 |
| | % LMW | 0.3 | 0.7 | 1.1 |
| Charge Variant Analysis | % Acidic | 12.1 | 26.0 | 35.8 |
| by CEX-UPLC | % Main | 35.3 | 22.3 | 14.8 |
| | % Basic | 52.6 | 51.7 | 49.4 |

TABLE 30
Research Stability of Formulation F2 Stored at 45° C.

| Formulation | | 100 mg/mL REGN1979, 10 mM | | |
|---|---|---|---|---|
| | | histidine, 5% Sorbitol, 0.1% | | |
| | | (w/v) polysorbate 20, pH 5.5 | | |
| Fill Volume | | 2.5 | | |
| | | Length of Storage at 45° C. (month) | | |
| Assay | | 0 | 0.5 | 1 |
| Color and Appearance | | Pass | Pass | Pass |
| Turbidity (Increase in OD | | 0.00 | 0.01 | 0.2 |
| at 405 nm) | | | | |
| pH | | 5.7 | 5.7 | 5.8 |
| Particulate Analysis | 2-10 μm | 140 | NR | 1685 |
| by MFI | ≥10 μm | 11 | NR | 40 |
| (Particles/mL) | ≥25 μm | 7 | NR | 13 |
| % Protein Recovered by | | 100 | 95 | 99 |
| RP-UPLC | | | | |
| Purity by SE-UPLC | % HMW | 2.4 | 4.5 | 7.4 |
| | % Native | 97.3 | 94.9 | 91.7 |
| | % LMW | 0.3 | 0.6 | 0.8 |
| Charge Variant | % Acidic | 12.5 | 29.6 | 37.6 |
| Analysis | % Main | 34.0 | 17.9 | 12.5 |
| by CEX-UPLC | % Basic | 53.5 | 52.5 | 49.9 |

TABLE 31
Research Stability of Formulation F3 Stored at 45° C.

| Formulation | | 100 mg/mL REGN1979, 10 mM | | |
|---|---|---|---|---|
| | | Succinate, 150 mM Sodium Chloride, | | |
| | | 0.1% (w/v) polysorbate 20, pH 6.0 | | |
| Fill Volume | | 2.5 | | |
| | | Length of Storage at 45° C. (month) | | |
| Assay | | 0 | 0.5 | 1 |
| Color and Appearance | | Pass | Fail | Fail |
| Turbidity | | 0.00 | 0.06 | 0.15 |
| (Increase in OD at 405 nm) | | | | |
| pH | | 5.9 | 5.9 | 6.0 |
| Particulate Analysis | 2-10 μm | 2256 | NR | 1299 |
| by MFI | ≥10 μm | 167 | NR | 50 |
| (Particles/mL) | ≥25 μm | 17 | NR | 15 |
| % Protein Recovered | | 100 | 96 | 98 |
| by RP-UPLC | | | | |
| Purity by SE-UPLC | % HMW | 2.7 | 8.2 | 13.8 |
| | % Native | 97.1 | 91.9 | 85.3 |
| | % LMW | 0.3 | 0.7 | 1.1 |
| Charge Variant | % Acidic | 12.8 | 25.3 | 36.4 |
| Analysis by | % Main | 33.5 | 21.9 | 14.2 |
| CEX-UPLC | % Basic | 53.7 | 52.9 | 49.4 |

TABLE 32
Research Stability of Formulation F4 Stored at 45° C.

| Formulation | | 100 mg/mL REGN1979, 10 mM | | |
|---|---|---|---|---|
| | | Acetate, 150 mM Arginine HCl, | | |
| | | 0.1% (w/v) polysorbate 80, pH 6.2 | | |
| Fill Volume | | 2.5 | | |
| | | Length of Storage at 45° C. (month) | | |
| Assay | | 0 | 0.5 | 1 |
| Color and Appearance | | Pass | Pass | Fail |
| Turbidity | | 0.00 | 0.04 | 0.08 |
| (Increase in OD at 405 nm) | | | | |
| pH | | 6.1 | 6.1 | 6.3 |
| Particulate Analysis by | 2-10 μm | 48 | NR | 1600 |
| MFI (particles/mL) | ≥10 μm | 11 | NR | 42 |
| | ≥25 μm | 0 | NR | 7 |
| % Protein Recovered by RP-UPLC | | 100 | 98 | 101 |
| Purity by SE-UPLC | % HMW | 2.6 | 7.4 | 13.0 |
| | % Native | 97.1 | 91.9 | 85.9 |
| | % LMW | 0.3 | 0.7 | 1.1 |
| Charge Variant Analysis | % Acidic | 12.2 | 25.5 | 36.0 |
| by CEX-UPLC | % Main | 34.2 | 22.9 | 51.6 |
| | % Basic | 36.0 | 14.8 | 49.2 |

TABLE 33
Research Stability of Formulation F5 Stored at 45° C.

| Formulation | | 100 mg/mL REGN1979, 10 mM | | |
|---|---|---|---|---|
| | | Acetate, 5% Sorbitol, 0.1% (w/v) | | |
| | | Poloxmer 188, pH 5.7 | | |
| Fill Volume | | 2.5 | | |
| | | Length of Storage at 45° C. (month) | | |
| Assay | | 0 | 0.5 | 1 |
| Color and Appearance | | Pass | Pass | Pass |
| Turbidity | | 0.00 | 0.01 | 0.01 |
| (Increase in OD at 405 nm) | | | | |
| pH | | 5.7 | 5.8 | 5.7 |
| Particulate Analysis by | 2-10 μm | 40 | NR | 1798 |
| MFI (Particles/mL) | ≥10 μm | 3 | NR | 25 |
| | ≥25 μm | 3 | NR | 3 |
| % Protein Recovered by RP-UPLC | | 100 | 96 | 99 |
| Purity by SE-UPLC | % HMW | 2.5 | 4.3 | 6.6 |
| | % Native | 97.2 | 95.2 | 92.7 |
| | % LMW | 0.3 | 0.5 | 0.8 |
| Charge Variant Analysis | % Acidic | 12.6 | 26.4 | 37.8 |
| by CEX-UPLC | % Main | 32.6 | 22.4 | 12.1 |
| | % Basic | 54.8 | 51.3 | 50.1 |

TABLE 34
Research Stability of Formulation F6 Stored at 45° C.

| Formulation | | 100 mg/mL REGN1979, 10 mM | | |
|---|---|---|---|---|
| | | histidine, 150 mM Sodium Chloride, | | |
| | | 0.1% (w/v) polysorbate 80, pH 5.5 | | |
| Fill Volume | | 2.5 | | |
| | | Length of Storage at 45° C. (month) | | |
| Assay | | 0 | 0.5 | 1 |
| Color and Appearance | | Pass | Fail | Fail |
| Turbidity | | 0.00 | 0.08 | 0.21 |
| (Increase in OD at 405 nm) | | | | |
| pH | | 5.7 | 5.8 | 5.8 |
| Particulate Analysis by | 2-10 μm | 459 | NR | ND |
| MFI (Particles/mL) | ≥10 μm | 13 | NR | ND |
| | ≥25 μm | 0 | NR | ND |
| % Protein Recovered by RP-UPLC | | 100 | 96 | 99 |
| Purity by SE-UPLC | % HMW | 2.5 | 4.3 | 16.7 |
| | % Native | 97.1 | 95.0 | 82.4 |
| | % LMW | 0.3 | 0.6 | 0.9 |
| Charge Variant Analysis | % Acidic | 12.7 | 27.8 | 37.3 |
| by CEX-UPLC | % Main | 32.7 | 20.3 | 12.6 |
| | % Basic | 54.6 | 51.9 | 50.1 |

TABLE 35
Research Stability of Formulation F7 Stored at 45° C.

| Formulation | | 100 mg/mL REGN1979, 10 mM | | |
|---|---|---|---|---|
| | | histidine, 5% Sorbitol, 0.1% (w/v) | | |
| | | polysorbate 80, pH 6.0 | | |
| Fill Volume | | 2.5 | | |
| | | Length of Storage at 45° C. (month) | | |
| Assay | | 0 | 0.5 | 1 |
| Color and Appearance | | Pass | Pass | Pass |
| Turbidity | | 0.00 | 0.01 | 0.02 |
| (Increase in OD at 405 nm) | | | | |
| pH | | 6.1 | 6.2 | 6.2 |
| Particulate | 2-10 μm | 71 | NR | 3745 |
| Analysis by MFI | ≥10 μm | 3 | NR | 15 |
| (particles/mL) | ≥25 μm | 3 | NR | 0 |
| % Protein Recovered RP-UPLC | | 100 | 96 | 101 |
| Purity by | % HMW | 2.5 | 3.9 | 6.1 |
| SE-UPLC | % Native | 97.1 | 95.6 | 93.1 |
| | % LMW | 0.3 | 0.5 | 0.8 |
| Charge Variant Analysis | % Acidic | 13.3 | 25.9 | 36.0 |
| by CEX-UPLC | % Main | 32.6 | 22.9 | 14.1 |
| | % Basic | 54.1 | 51.2 | 49.9 |

TABLE 36
Research Stability of Formulation F8 Stored at 45° C.

| Formulation | | 100 mg/mL REGN1979, 10 mM | | |
|---|---|---|---|---|
| | | Succinate, 150 mM Arginine HCI, | | |
| | | 0.1% (w/v) poloxmer 188, pH 5.5 | | |
| Fill Volume | | 2.5 | | |
| | | Length of Storage at 45° C. (month) | | |
| Assay | | 0 | 0.5 | 1 |
| Color and Appearance | | Pass | Fail | Fail |
| Turbidity | | 0.00 | 0.09 | 0.24 |
| (Increase in OD at 405 nm) | | | | |
| pH | | 5.5 | 5.5 | 5.5 |
| Particulate | 2-10 μm | 171 | NR | 1468 |
| Analysis by MFI | ≥10 μm | 3 | NR | 15 |
| (particles/mL) | ≥25 μm | 0 | NR | 7 |
| % Protein Recovered | | 100 | 93 | 95 |
| by RP-UPLC | | | | |
| Purity by SE-UPLC | % HMW | 2.4 | 3.9 | 28.1 |
| | % Native | 97.3 | 95.3 | 71.0 |
| | % LMW | 0.3 | 0.6 | 0.9 |
| Charge Variant | % Acidic | 12.7 | 30.1 | 39.7 |
| Analysis | % Main | 32.5 | 17.3 | 10.5 |
| by CEX-UPLC | % Basic | 54.8 | 52.6 | 49.9 |

TABLE 37
Research Stability of Formulation F9 Stored at 45° C.

| Formulation | | 100 mg/mL REGN1979, 10 mM | | |
|---|---|---|---|---|
| | | Succinate, 5% Sorbitol, 0.1% (w/v) | | |
| | | polysorbate 20, pH 6.5 | | |
| Fill Volume | | 2.5 | | |
| | | Length of Storage at 45° C. (month) | | |
| Assay | | 0 | 0.5 | 1 |
| Color and Appearance | | Pass | Pass | Pass |
| Turbidity | | 0.00 | 0.02 | 0.04 |
| (Increase in OD at 405 nm) | | | | |
| pH | | 6.3 | 6.3 | 6.4 |
| Particulate Analysis by | 2-10 μm | 272 | NR | 309 |
| MFI (particles/mL) | ≥10 μm | 34 | NR | 19 |
| | ≥25 μm | 13 | NR | 15 |
| % Protein Recovered | | 100 | 99 | 105 |
| by RP-UPLC | | | | |
| Purity by SE-UPLC | % HMW | 3.0 | 5.1 | 7.8 |
| | % Native | 96.8 | 94.4 | 91.4 |
| | % LMW | 0.3 | 0.6 | 0.8 |
| Charge Variant | % Acidic | 13.4 | 27.9 | 36.7 |
| Analysis | % Main | 32.6 | 18.9 | 14.3 |
| by CEX-UPLC | % Basic | 54.0 | 53.2 | 49.0 |

TABLE 38
Research Stability of Formulation F10 Stored at 45° C.

| Formulation | | 100 mg/mL REGN1979, 10 mM | | |
|---|---|---|---|---|
| | | Acetate, 150 mM Sodium Chloride, | | |
| | | 0.1% (w/v) poloxmer 188, pH 6.2 | | |
| Fill Volume | | 2.5 | | |
| | | Length of Storage at 45° C. (month) | | |
| Assay | | 0 | 0.5 | 1 |
| Color and Appearance | | Pass | Pass | Fail |
| Turbidity | | 0.00 | 0.05 | 0.10 |
| (Increase in OD at 405 nm) | | | | |
| pH | | 6.2 | 6.2 | 6.3 |
| Particulate Analysis by | 2-10 μm | 132 | NR | 1691 |
| MFI (Particles/mL) | ≥10 μm | 11 | NR | 32 |
| | ≥25 μm | 3 | NR | 3 |
| % Protein Recovered by RP-UPLC | | 100 | 97 | 101 |
| Purity by SE-UPLC | % HMW | 3.0 | 6.9 | 11.0 |
| | % Native | 96.7 | 92.4 | 88.2 |
| | % LMW | 0.3 | 0.7 | 0.9 |
| Charge Variant Analysis | % Acidic | 13.6 | 26.4 | 35.8 |
| by CEX-UPLC | % Main | 31.8 | 21.0 | 14.4 |
| | % Basic | 54.6 | 52.5 | 49.9 |

TABLE 39
Research Stability of Formulation F11 Stored at 45° C.

| Formulation | | 100 mg/mL REGN1979, 10 mM | | |
|---|---|---|---|---|
| | | Acetate, 10% Sucrose, 0.1% (w/v) | | |
| | | polysorbate 20, pH 5.2 | | |
| Fill Volume | | 2.5 | | |
| | | Length of Storage at 45° C. (month) | | |
| Assay | | 0 | 0.5 | 1 |
| Color and Appearance | | Pass | Pass | Pass |
| Turbidity | | 0.00 | 0.01 | 0.09 |
| (Increase in OD at 405 nm) | | | | |
| pH | | 5.2 | 5.2 | 5.3 |
| Particulate Analysis by | 2-10 μm | 184 | NR | 1091 |
| MFI (Particles/mL) | ≥10 μm | 17 | NR | 5 |
| | ≥25 μm | 7 | NR | 0 |
| % Protein Recovered by RP-UPLC | | 100 | 96 | 97 |
| Purity by | % HMW | 2.4 | 5.2 | 9.0 |
| SE-UPLC | % Native | 97.4 | 94.2 | 90.1 |
| | % LMW | 0.3 | 0.6 | 0.9 |
| Charge Variant Analysis | % Acidic | 13.4 | 32.6 | 41.9 |
| by CEX-UPLC | % Main | 33.0 | 16.5 | 10.1 |
| | % Basic | 53.6 | 50.9 | 48.1 |

TABLE 40
Research Stability of Formulation F12 Stored at 45° C.

| Formulation | | 100 mg/mL REGN1979, 10 mM | | |
|---|---|---|---|---|
| | | histidine, 10% Sucrose, 0.1% (w/v) | | |
| | | poloxmer 188, pH 6.5 | | |
| Fill Volume | | 2.5 | | |
| | | Length of Storage at 45° C. month) | | |
| Assay | | 0 | 0.5 | 1 |
| Color and Appearance | | Pass | Pass | Pass |
| Turbidity | | 0.00 | 0.01 | 0.03 |
| (Increase in OD at 405 nm) | | | | |
| pH | | 6.5 | 6.6 | 6.6 |
| Particulate Analysis by | 2-10 μm | 249 | NR | 1639 |
| MFI (Particles/mL) | ≥10 μm | 55 | NR | 50 |
| | ≥25 μm | 11 | NR | 11 |
| % Protein Recovered by RP-UPLC | | 100 | 99 | 104 |
| Purity by SE-UPLC | % HMW | 2.7 | 3.8 | 5.7 |
| | % Native | 97.0 | 95.6 | 93.5 |
| | % LMW | 0.3 | 0.6 | 0.9 |
| Charge Variant Analysis | % Acidic | 13.1 | 27.4 | 38.0 |
| by CEX-UPLC | % Main | 33.6 | 22.3 | 14.4 |
| | % Basic | 53.3 | 50.3 | 47.6 |

TABLE 41
Research Stability of Formulation F13 Stored at 45° C.

| Formulation | | 100 mg/mL REGN1979, | | |
|---|---|---|---|---|
| | | 10 mM Succinate, | | |
| | | 10% Sucrose, 0.1% (w/v) | | |
| | | polysorbate 80, pH 6.0 | | |
| Fill Volume | | 2.5 | | |
| | | Length of Storage at 45° C. (month) | | |
| Assay | | 0 | 0.5 | 1 |
| Color and Appearance | | Pass | Pass | Pass |
| Turbidity (Increase in OD at 405 nm) | | 0.00 | 0.01 | 0.01 |
| pH | | 5.9 | 5.9 | 6.0 |
| Particulate | 2-10 μm | 73 | NR | 1108 |
| Analysis by | ≥10 μm | 15 | NR | 55 |
| MFI (particles/mL) | ≥25 μm | 3 | NR | 9 |
| % Protein Recovered by RP-UPLC | | 100 | 96 | 101 |
| Purity by | % HMW | 2.6 | 4.2 | 6.4 |
| SE-UPLC | % Native | 97.1 | 95.2 | 92.8 |
| | % LMW | 0.3 | 0.6 | 0.8 |
| Charge | % Acidic | 12.8 | 25.9 | 37.8 |
| Variant | % Main | 32.8 | 22.9 | 13.9 |
| Analysis by CEX-UPLC | % Basic | 54.4 | 51.2 | 48.3 |

### Example 3: Viscosity of Formulations

Viscosity measurements were performed at 20° C. using as Rheosense m-VROC capillary viscometer (Rheosense, San Ramon, Calif.). Samples of various REGN1979 concentrations ranging from 79.9 to 184.9 mg/ml were prepared in a formulation buffer containing 10 mM histidine and 5% sucrose (pH 5.8). All samples were filtered using 2 μm centrifugal spin filters before measurement. The results of the measurements are shown below in Table 42.

TABLE 42
Viscosity-Protein Concentrations

| Protein Concentration (mg/ml) of drug | Viscosity at 20° C. |
|---|---|
| substance formulation (10 mM histidine, | without viscosity |
| pH 5.8, 5% sucrose) | reducer (cP) |
| 79.9 | 2.8 |
| 115.7 | 5.4 |
| 154.8 | 12.1 |
| 184.9 | 33.0 |

The drug product, containing a specific concentration of REGN1979, L-histidine (0.57 mg/ml), L-histidine monohydrochloride monohydrate (1.33 mg/ml), sucrose (100 mg/ml), polysorbate 80 (1 mg/ml), and water for injection (USP), was determined to have the viscosity shown in Table 43, below.

TABLE 43
Drug Product Viscosity

| | REGN1979 Concentration | Viscosity (cP) |
|---|---|---|
| 2 mg drug product | 2 mg/ml | 1.4 |
| 80 mg and 160 mg drug product | 20 mg/ml | 1.7 |
| 200 mg drug product | 100 mg/ml | 4.95 |

The low viscosity (e.g., less than 15 cP) observed in formulations containing as much as 154.8 mg/ml of antibody is advantageous for use in prefilled syringes or autoinjectors.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A stable liquid pharmaceutical formulation comprising:
(a) a bispecific antibody comprising a first antigen-binding domain that binds specifically to human CD20 and a second antigen-binding domain that binds specifically to human CD3, wherein the first antigen-binding domain comprises three heavy chain complementarity determining regions (CDRs) (A1-HCDR1, A1-HCDR2 and A1-HCDR3) contained in a heavy chain variable region (HCVR) and three light chain CDRs (LCDR1, LCDR2 and LCDR3) contained in a light chain variable region (LCVR), and the second antigen-binding domain comprises three heavy chain CDRs (A2-HCDR1, A2-HCDR2 and A2-HCDR3) contained in a heavy chain variable region (HCVR) and three light chain CDRs (LCDR1, LCDR2 and LCDR3) contained in a light chain variable region (LCVR), wherein A1-HCDR1, A1-HCDR2 and A1-HCDR3 comprise the amino acid sequences, respectively, of SEQ ID NOs: 7, 8 and 9, A2-HCDR1, A2-HCDR2 and A2-HCDR3 comprise the amino acid sequences, respectively, of SEQ ID NOs: 10, 11 and 12, and LCDR1, LCDR2 and LCDR3 comprise the amino acid sequences, respectively, of SEQ ID NOs: 13, 14 and 15;
(b) a buffer comprising histidine;
(c) an organic co-solvent comprising polysorbate; and
(d) a stabilizer comprising a sugar;
wherein the formulation has a pH of 5.8±0.3.

2. The pharmaceutical formulation of claim 1, wherein the antibody concentration is from 1 mg/ml±0.1 mg/ml to 200 mg/ml±20 mg/ml.

3-8. (canceled)

9. The pharmaceutical formulation of claim 2, wherein the histidine buffer concentration is from 5 mM±1 mM to 15 mM±3 mM.

10. (canceled)

11. The pharmaceutical formulation of claim 9, wherein the histidine buffer comprises L-histidine and L-histidine monohydrochloride monohydrate.

12. (canceled)

13. The pharmaceutical formulation of claim 9, wherein the polysorbate concentration is from 0.01%±0.005% to 0.5%±0.25% w/v.

14-15. (canceled)

16. The pharmaceutical formulation of claim 13, wherein the polysorbate is polysorbate 80.

17. The pharmaceutical formulation of claim 16, wherein the sugar is sucrose.

18. (canceled)

19. The pharmaceutical formulation of claim 17, wherein the sucrose concentration is from 8%±0.5% to 12%±0.5% w/v.

20-21. (canceled)

22. The pharmaceutical formulation of claim 1 comprising:
(a) 2 mg/ml±0.2 mg/ml antibody,
(b) 10 mM±1 mM histidine buffer,
(c) 0.1%±0.05% w/v polysorbate, and
(d) 10%±1% w/v sucrose,
at pH 5.8±0.3.

23-24. (canceled)

25. The pharmaceutical formulation of claim 1 comprising:
(a) 20 mg/ml±2 mg/ml antibody,
(b) 10 mM±1 mM histidine buffer,
(c) 0.1%±0.05% w/v polysorbate, and
(d) 10%±1% w/v sucrose,
at pH 5.8±0.3.

26-27. (canceled)

28. The pharmaceutical formulation of claim 1 comprising:
(a) 80 mg/ml±8 mg/ml antibody,
(b) 10 mM±1 mM histidine buffer,
(c) 0.1%±0.05% w/v polysorbate, and
(d) 10%±1% w/v sucrose,
at pH 5.8±0.3.

29-30. (canceled)

31. The pharmaceutical formulation of claim 1 comprising:
(a) 100 mg/ml±10 mg/ml antibody,
(b) 10 mM±1 mM histidine buffer,
(c) 0.1%±0.05% w/v polysorbate, and
(d) 10%±1% w/v sucrose,
at pH 5.8±0.3.

32-33. (canceled)

34. The pharmaceutical formulation of claim 1 comprising:
(a) 160 mg/ml±16 mg/ml antibody,
(b) 10 mM±1 mM histidine buffer,
(c) 0.1%±0.05% w/v polysorbate, and
(d) 10%±1% w/v sucrose,
at pH 5.8±0.3.

35-36. (canceled)

37. The pharmaceutical formulation of claim 19, wherein at least 90% of the antibody has native conformation after 1 month of storage at 45° C., as determined by size exclusion-ultra performance liquid chromatography (SE-UPLC).

38. The pharmaceutical formulation of claim 37, wherein at least 93% of the antibody has native conformation after 1 month of storage at 45° C., as determined by SE-UPLC.

39. The pharmaceutical formulation of claim 38, wherein at least 95% of the antibody has native conformation after 1 month of storage at 45° C., as determined by SE-UPLC.

40. The pharmaceutical formulation of claim 19, wherein at least 95% of the antibody has native conformation after 3 months of storage at 25° C. and 60% relative humidity, as determined by SE-UPLC.

41. The pharmaceutical formulation of claim 40, wherein at least 97% of the antibody has native conformation after 3 months of storage at 25° C. and 60% relative humidity, as determined by SE-UPLC.

42. The pharmaceutical formulation of claim 19, wherein at least 95% of the antibody has native conformation after 3 months of storage at 5° C., as determined by SE-UPLC.

43. The pharmaceutical formulation of claim 42, wherein at least 98% of the antibody has native conformation after 3 months of storage at 5° C., as determined by SE-UPLC.

44. The pharmaceutical formulation of claim 19, wherein the formulation contains no more than 6% high molecular weight (HMW) species after 1 month of storage at 45° C., as determined by SE-UPLC.

45. The pharmaceutical formulation of claim 44, wherein the formulation contains no more than 5.6% HMW species after 1 month of storage at 45° C., as determined by SE-UPLC.

46. The pharmaceutical formulation of claim 19, wherein the formulation contains no more than 2% HMW species after 3 months of storage at 25° C. and 60% relative humidity, as determined by SE-UPLC.

47. The pharmaceutical formulation of claim 46, wherein the formulation contains no more than 1% HMW species after 3 months of storage at 25° C. and 60% relative humidity, as determined by SE-UPLC.

48. The pharmaceutical formulation of claim 19, wherein the formulation contains no more than 1.5% HMW species after 3 months of storage at 5° C., as determined by SE-UPLC.

49. The pharmaceutical formulation of claim 48, wherein the formulation contains no more than 1% HMW species after 9 months of storage at 5° C., as determined by SE-UPLC.

50-53. (canceled)

54. The pharmaceutical formulation of claim 19, wherein the antibody comprises a human IgG heavy chain constant region attached, respectively, to the HCVR of each of the first antigen-binding domain and the second antigen-binding domain.

55. The pharmaceutical formulation of claim 54, wherein the heavy chain constant region is of isotype IgG1.

56. The pharmaceutical formulation of claim 54, wherein the heavy chain constant region is of isotype IgG4.

57-58. (canceled)

59. The pharmaceutical formulation of claim 54, wherein the modification comprises a H435R substitution and a Y436F substitution (EU numbering) in a heavy chain of isotype IgG1 or IgG4.

60. The pharmaceutical formulation of claim 54, wherein the antibody comprises a heavy chain constant region comprising an amino acid sequence selected from the group consisting of SEQ ID NO: 16, SEQ ID NO: 17, SEQ ID NO: 18, and SEQ ID NO: 19.

61. The pharmaceutical formulation of claim 60, wherein the antibody comprises a heavy chain constant region comprising the amino acid sequence of SEQ ID NO: 16 and a heavy chain constant region comprising the amino acid sequence of SEQ ID NO: 17.

62. The pharmaceutical formulation of claim 60, wherein the antibody comprises a heavy chain constant region comprising the amino acid sequence of SEQ ID NO: 18 and a heavy chain constant region comprising the amino acid sequence of SEQ ID NO: 19.

63. The pharmaceutical formulation of claim 1, wherein the antibody comprises a first heavy chain containing the HCVR of the first antigen-binding domain and a second heavy chain containing the HCVR of the second antigen-binding domain, wherein the first heavy chain comprises residues 1-452 of the amino acid sequence of SEQ ID NO: 1 and the second heavy chain comprises residues 1-448 of the amino acid sequence of SEQ ID NO: 2.

64. The pharmaceutical formulation of claim 63, wherein the antibody comprises a common light chain containing the LCVR of the first and second antigen-binding domains, wherein the common light chain comprises the amino acid sequence of SEQ ID NO: 3.

65. A pharmaceutical formulation comprising:
(a) 2 mg/ml±0.2 mg/ml of a bispecific antibody comprising a first antigen-binding domain that binds specifically to human CD20 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6;
(b) 10 mM±1 mM histidine buffer, pH 5.8±0.2,
(c) 0.1%±0.01% w/v polysorbate 80, and
(d) 10%±1% w/v sucrose.

66-67. (canceled)

68. A pharmaceutical formulation comprising:
(a) 20 mg/ml±2 mg/ml of a bispecific antibody comprising a first antigen-binding domain that binds specifically to human CD20 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6;
(b) 10 mM±1 mM histidine buffer, pH 5.8±0.2,
(c) 0.1%±0.01% w/v polysorbate 80, and
(d) 10%±1% w/v sucrose.

69-70. (canceled)

71. A pharmaceutical formulation comprising:
(a) 100 mg/ml±10 mg/ml of a bispecific antibody comprising a first antigen-binding domain that binds specifically to human CD20 and a second antigen-binding domain that binds specifically to human CD3, wherein the first antigen-binding domain comprises a HCVR comprising the amino acid sequence of SEQ ID NO: 4 and a LCVR comprising the amino acid sequence of SEQ ID NO: 6, and the second antigen-binding domain comprises a HCVR comprising the amino acid sequence of SEQ ID NO: 5 and a LCVR comprising the amino acid sequence of SEQ ID NO: 6;
(b) 10 mM±1 mM histidine buffer, pH 5.8±0.2,
(c) 0.1%±0.01% w/v polysorbate 80, and
(d) 10%±1% w/v sucrose.

72-84. (canceled)

85. The pharmaceutical formulation of claim 68, wherein: (a) at least 90% of the antibody has native conformation after 1 month of storage at 45° C.; (b) at least 93% of the antibody has native conformation after 1 month of storage at 45° C.; (c) at least 95% of the antibody has native conformation after 1 month of storage at 45° C.; (d) at least 95% of the antibody has native conformation after 3 months of storage at 25° C. and 60% relative humidity; (e) at least 97% of the antibody has native conformation after 3 months of storage at 25° C. and 60% relative humidity; (f) at least 95% of the antibody has native conformation after 3 months of storage at 5° C.; (g) at least 98% of the antibody has native conformation after 3 months of storage at 5° C.; (h) the formulation contains no more than 6% high molecular weight (HMW) species after 1 month of storage at 45° C.; (i) the formulation contains no more than 5.6% HMW species after 1 month of storage at 45° C.; (j) the formulation contains no more than 2% HMW species after 3 months of storage at 25° C. and 60% relative humidity; (k) the formulation contains no more than 1% HMW species after 3 months of storage at 25° C. and 60% relative humidity; (1) the formulation contains no more than 1.5% HMW species after 3 months of storage at 5° C.; and/or (m) the formulation contains no more than 1% BMW species after 9 months of storage at 5° C.;
as determined by SE-UPLC

86. A pharmaceutical composition, wherein the composition comprises the pharmaceutical formulation of claim 19, and the composition is contained in a container.

87-92. (canceled)

93. The pharmaceutical composition of claim 86, wherein the container comprises a headspace comprising a gas, wherein the gas comprises less than 5% oxygen by volume.

94. The pharmaceutical composition of claim 93, wherein the gas comprises less than 1% oxygen by volume, or no more than 0.1% oxygen by volume.

95. A kit comprising (i) a container containing a composition comprising the pharmaceutical formulation of claim 19, and instructions for use of the composition.

96-102. (canceled)

103. A unit dosage form comprising the pharmaceutical formulation of claim 19, wherein the antibody is present in an amount of from 0.1 mg to 500 mg.

104-116. (canceled)

117. A container containing a pharmaceutical composition, wherein the composition comprises the pharmaceutical formulation of claim 19.

118-121. (canceled)
